(19) United States
(12) Patent Application Publication
(10) Pub. No.: US 2009/0305089 A1
(43) Pub. Date: Dec. 10, 2009

(54) ORGANELLES IN BIOANODES, BIOCATHODES, AND BIOFUEL CELLS

(75) Inventors: Shelley D. Minteer, Pacific, MO (US); Robert Arechederra, St. Louis, MO (US)

Correspondence Address: SENNIGER POWERS LLP, 100 NORTH BROADWAY, 17TH FLOOR, ST LOUIS, MO, 63102, US

(73) Assignee: AKERMIN, INC., St. Louis, MO (US)

(21) Appl. No.: 12/373,777

(22) PCT Filed: Jul. 16, 2007
(86) PCT No.: PCT/US07/73596

§ 371 Date: Apr. 24, 2009

Related U.S. Application Data
(60) Provisional application No. 60/807,419, filed on Jul. 14, 2006.

(51) Int. Cl. H01M 8/16; H01M 8/04; C12N 1/00

(52) U.S. Cl. 429/13; 429/43; 435/317.1

(57) ABSTRACT

Bioanodes, biocathodes, and biofuel cells comprising an electron conductor, at least one anode organelle or cathode organelle, and an organelle immobilization material. The anode organelle is capable of reacting with a fuel fluid to produce an oxidized form of the fuel fluid, and capable of releasing electrons to the electron conductor. The cathode organelle is capable of reacting with an oxidant to produce water, and capable of gaining electrons from the electron conductor. The organelle immobilization material for both the anode organelle and the cathode organelle is capable of immobilizing the organelle, and is permeable to the fuel fluid and/or the oxidant. In various embodiments, the organelle immobilization material is further capable of stabilizing the organelle.

This invention was made with Government support under Grant No. 3-00487 awarded by the Department of Defense. The Government has certain rights in the invention.

### BACKGROUND OF THE INVENTION

The present invention is directed in general to biological organelle-based fuel cells (a.k.a. biofuel cells) and their methods of manufacture and use. More specifically, the invention is directed to bioelectrodes, bioanodes, biocathodes, and biofuel cells comprising organelles capable of providing transfer of electrons between the fuel fluid and electron conductor, and their method of manufacture and use.

A biofuel cell is an electrochemical device in which energy derived from chemical reactions is converted to electrical energy by means of the catalytic activity of living cells and/or their enzymes. Biofuel cells generally use complex molecules to generate at the anode the hydrogen ions required to reduce oxygen to water, while generating free electrons for use in electrical applications. A bioanode is the electrode of the biofuel cell where electrons are released upon the oxidation of a fuel and a biocathode is the electrode where electrons and protons from the anode are used by the catalyst to reduce oxygen to water. Biofuel cells differ from the traditional fuel cell by the material used to catalyze the electrochemical reaction. Rather than using precious metals as catalysts, biofuel cells rely on biological molecules such as enzymes to carry out the reactions.

In some cases, organelle immobilization may provide advantages over enzyme isolation and immobilization in some bioelectrodes.

### SUMMARY OF THE INVENTION

Among the various aspects of the invention are bioelectrodes, bioanodes, biocathodes, immobilization materials and biofuel cells comprising organelles.

Another aspect is a bioelectrode comprising an electron conductor, at least one organelle comprising at least one enzyme, and an organelle immobilization material. The organelle immobilization material is capable of immobilizing the organelle.

Yet another aspect is a bioanode comprising an electron conductor, at least one anode organelle comprising at least one enzyme, and an organelle immobilization material. The enzyme is capable of reacting with a fuel fluid to produce an oxidized form of the fuel fluid and of releasing electrons to the electron conductor. The organelle immobilization material is capable of immobilizing the organelle and is permeable to the fuel fluid.

A further aspect of the invention is a bioanode comprising an electron conductor, at least one anode organelle comprising at least one enzyme, and an organelle immobilization material. The enzyme is capable of reacting with an oxidized form of an electron mediator and a fuel fluid to produce an oxidized form of the fuel fluid and a reduced form of the electron mediator. The reduced form of the electron mediator is capable of releasing electrons to the electron conductor. The organelle immobilization material is capable of immobilizing the organelle and is permeable to the fuel fluid.

Yet another aspect is a bioanode comprising an electron conductor, at least one anode organelle comprising at least one enzyme, an organelle immobilization material, and an electrocatalyst adjacent the electron conductor. The enzyme is capable of reacting with an oxidized form of the electron mediator and a fuel fluid to produce an oxidized form of the fuel fluid and a reduced form of the electron mediator. The organelle immobilization material is capable of immobilizing the organelle and is permeable to the fuel fluid. An oxidized form of the electrocatalyst is capable of reacting with the reduced form of the electron mediator to produce an oxidized form of the electron mediator and a reduced form of the electrocatalyst, and the reduced form of the electrocatalyst is capable of releasing electrons to the electron conductor.

A further aspect is a biocathode comprising an electron conductor, at least one cathode organelle comprising at least one enzyme, and an organelle immobilization material. The enzyme is capable of reacting with an oxidant to produce water and gaining electrons from the electron conductor. The organelle immobilization material is capable of immobilizing the organelle and is permeable to the oxidant.

Yet another aspect is a biocathode comprising an electron conductor, at least one cathode organelle comprising at least one enzyme, and an organelle immobilization material. The enzyme is capable of reacting with a reduced form of an electron mediator and an oxidant to produce an oxidized form of the electron mediator and water. The oxidized form of the electron mediator is capable of gaining electrons from the electron conductor to produce the reduced form of the electron mediator. The organelle immobilization material is capable of immobilizing the organelle and is permeable to the oxidant.

Another aspect of the invention is a biocathode comprising an electron conductor, at least one cathode organelle comprising at least one enzyme, an organelle immobilization material, and an electrocatalyst adjacent the electron conductor. The enzyme is capable of reacting with a reduced form of an electron mediator and an oxidant to produce an oxidized form of the electron mediator and water. The organelle immobilization material capable of immobilizing the organelle and is permeable to the oxidant. An oxidized form of the electrocatalyst is capable of gaining electrons from the electron conductor to produce a reduced form of the electrocatalyst that is capable of reacting with an oxidized form of the electron mediator to produce a reduced form of the electron mediator and an oxidized form of the electrocatalyst.

A further aspect is an organelle immobilized in a non-naturally occurring colloidal immobilization material. The immobilization material is capable of immobilizing the organelle and is permeable to a compound smaller than the organelle.

Yet another aspect is an organelle immobilized in an acellular, colloidal immobilization material. The immobilization material is capable of immobilizing the enzyme and is permeable to a compound smaller than the organelle.

Another aspect is an organelle immobilized in a micellar or inverted micellar immobilization material. The immobilization material is capable of immobilizing the organelle and is permeable to a compound smaller than the organelle.

Yet another aspect is a biofuel cell wherein the organelle is mitochondria, the fuel fluid is pyruvate, and the bioanode further comprises an agent which inhibits the enzyme from reacting with the fuel fluid until the mitochondria is exposed to a nitroaromatic explosive. From this biofuel cell, an alarm signal is produced when the nitroaromatic explosive is present and there is an alarm that detects the alarm signal and provides an alert to the presence of the explosive.

A further aspect is a method for detecting a nitroaromatic explosive using the biofuel cell described above comprising exposing the biofuel cell to the nitroaromatic explosive so that the enzyme will react with the fuel fluid and the biofuel cell will generate electricity to produce the alarm signal indicating that the nitroaromatic explosive has been detected.

Other objects and features will be in part apparent and in part pointed out hereinafter.

### DESCRIPTION OF THE DRAWINGS

FIG. 1 shows a representative power curve for a mitochondrial anode in 100 mM pyruvate fuel with pH 7.15 phosphate buffer and 1 M NaCl electrolyte.

FIG. 2 shows a representative power curve for a mitochondrial anode in 100 mM glucose fuel with pH 7.15 phosphate buffer and 1 M NaCl electrolyte.

FIG. 3 shows representative fluorescence intensity of luciferin luciferase for immobilized mitochondria in 1 mM pyruvate fuel with pH 7.15 phosphate buffer.

FIG. 4 shows a representative power curve for a mitochondrial biofuel cell in 100 mM pyruvate fuel with pH 7.15 phosphate buffer.

FIG. 5 is a schematic of an I-Cell as described in Example 5.

FIG. 6 is a representative steady state power curve for a low surface area mitochondria-modified electrode that exhibits direct electron transfer.

FIG. 7 is a representative peak power curve for a low surface area mitochondria-modified electrode that exhibits direct electron transfer.

FIG. 8 is a representative steady state power curve for a high surface area mitochondria-modified electrode that exhibits direct electron transfer.

FIG. 9 is a representative peak power curve for a high surface area mitochondria-modified electrode that exhibits direct electron transfer.

FIG. 10 shows cyclic voltammograms (CV) of a mitochondria-modified electrode. One CV is in the presence of pyruvate fuel and in the other CV, pyruvate fuel is absent.

FIG. 11 is a representative power curve for a pyruvate biofuel cell with uninhibited mitochondria immobilized in tetrabutylammonium-modified Nafion®.

FIG. 12 is a representative power curve for a pyruvate biofuel cell with an anode consisting of a mitochondria-modified electrode that has been inhibited with oligomycin.

FIG. 13 is a representative power curve for a pyruvate biofuel cell with an anode consisting of a mitochondria-modified electrode that has been inhibited with oligomycin and soaked in nitrobenzene solution.

FIG. 14 is a graph of absorbance at 622 nm vs. time of illumination with light for a thylakoid immobilized in a hexanal modified chitosan polymer in a solution containing 2.5 mL buffer and 200 μL of 500 μM dichlorophenolindophenol (DCPIP).

FIG. 15 is a graph of absorbance at 622 nm vs. time of illumination with light for a thylakoid immobilized in a TBAB-modified Nafion® polymer in a solution containing 2.5 mL buffer and 200 μL of 500 μM dichlorophenolindophenol (DCPIP).

FIG. 16 is a schematic of a self-powered nitroaromatic explosives sensor.

FIG. 17 shows a single, functional bioanode or biocathode.

FIG. 18 shows a microfluidic biofuel cell.

FIG. 19(a)-(d) shows the procedure for forming a single microelectrode.

FIG. 20 shows a microfluidic biofuel cell stack.

### DETAILED DESCRIPTION OF THE INVENTION

The present invention is directed to bioelectrodes, bioanodes, biocathodes, and/or biofuel cells comprising at least one organelle which comprises at least one enzyme capable of electron transfer with an electron conductor. In various embodiments, the organelle comprises an electron mediator and/or an electrocatalyst in addition to the enzyme. In these embodiments, the electron transfer from the fuel fluid to the electron conductor is mediated by the electron mediator and/or electrocatalyst. Among the advantages of immobilized organelles in bioanodes, biocathodes, and biofuel cells is that all the enzymes of interest are present in the organelle in the appropriate concentration and geometric configuration for maximum enzyme activity. In various embodiments, the bioanodes or biocathodes can comprise either immobilized organelles and/or immobilized enzymes. For example, a biofuel cell can contain either an anode or a bioanode comprising either an immobilized enzyme or an immobilized organelle, and either a cathode or a biocathode comprising either an immobilized enzyme or an immobilized organelle.

Generally, another aspect of the invention is a bioelectrode comprising an electron conductor, at least one organelle, and an organelle immobilization material, wherein the organelle immobilization material immobilizes the organelle at the surface of the electron conductor. The organelle is isolated from a cell or the organelle immobilization material is non-microbial. In particular, such a bioelectrode comprises an electron conductor, at least one mitochondria or mitoplasts, and a mitochondrion immobilization material, wherein the mitochondrion immobilization material immobilizes the mitochondrion at the surface of the electron conductor.

In yet a further embodiment, the bioelectrode assembly of the present invention has increased enzyme stability because the enzymes within the organelle are capable of regenerating. For example, upon immobilization of mitochondria in a bioanode, the enzymes contained within the mitochondria are able to regenerate, and thus, are more stable than they would be if immobilized in an isolated form. For use in a biocathode or a bioanode, the immobilization material, which is preferably non-microbial, forms a barrier that provides mechanical and chemical stability to the organelle. For purposes of the present invention, an enzyme or organelle is “stabilized” if the enzyme retains at least about 75% of its initial catalytic activity upon continuous operation in a biofuel cell for at least about 7 days to about 730 days.

### I. Biofuel Cell

Among the various aspects of the invention is a biofuel cell utilizing a fuel fluid to produce electricity via enzyme mediated redox reactions taking place at an electrode with at least one immobilized organelle therein. Depending on the particular chemical transformations carried out at the anode and the cathode, the biofuel cell can contain bioanodes or biocathodes comprising either immobilized enzymes or immobilized organelles.

As in a standard electrochemical cell, the anode is the site for an oxidation reaction of a fuel fluid with a concurrent release of electrons. The electrons are directed from the anode through an electrical connector to some power consuming device. The electrons move through the device to another electrical connector, which transports the electrons to the biofuel cell's biocathode where the electrons are used to reduce an oxidant to produce water. In this manner, the biofuel cell of the present invention acts as an energy source (electricity) for an electrical load external thereto. To facilitate the fuel fluid's redox reactions, the electrodes comprise an electron conductor, an organelle, optionally, an electron mediator, and an organelle immobilization material.

In accordance with the invention, an electron mediator or an electron mediator and an electrocatalyst can be included in the bioelectrode external to the organelle. In various embodiments, this electron mediator and/or electrocatalyst external to the organelle is in addition to the electron mediator and/or electrocatalyst within the organelle. The electron mediator is a compound that can accept electrons or donate electrons. In a presently preferred biofuel cell, the oxidized form of the electron mediator reacts with the fuel fluid and the enzyme to produce the oxidized form of the fuel fluid and the reduced form of the electron mediator at the bioanode. Subsequently or concurrently, the reduced form of the electron mediator reacts with the oxidized form of the electrocatalyst to produce the oxidized form of the electron mediator and the reduced form of the electrocatalyst. The reduced form of the electrocatalyst is then oxidized at the bioanode and produces electrons to generate electricity. The redox reactions at the bioanode, except the oxidation of the fuel fluid, can be reversible, so the enzyme, electron mediator and electrocatalyst are not consumed. Optionally, these redox reactions can be irreversible if an electron mediator and/or an electrocatalyst is added to provide additional reactant.

Alternatively, an electron conductor and an organelle can be used wherein an electron mediator in contact with the bioanode is able to transfer electrons between its oxidized and reduced forms at unmodified electrodes. If the electron mediator is able to transfer electrons between its oxidized and reduced forms at an unmodified bioanode, the subsequent reaction between the electrocatalyst and the electron mediator is not necessary and the electron mediator itself is oxidized at the bioanode to produce electrons and thus, electricity. Further, in other embodiments, an electron conductor, organelle, and fuel fluid can be used wherein an enzyme within the organelle is able to transfer electrons to the electron conductor.

At the biocathode, electrons originating from the bioanode flow into the biocathode's electron conductor. There, the electrons combine with an oxidized form of an electrocatalyst, which is in contact with the electron conductor. This reaction produces a reduced form of the electrocatalyst, which in turn reacts with an oxidized form of an electron mediator to produce a reduced form of the electron mediator and an oxidized form of the electrocatalyst. Next, the reduced form of the electron mediator reacts with an oxidized form of the oxidant to produce an oxidized form of the electron mediator and water. In one embodiment, an organelle immobilization material permeable to the oxidant is present, which comprises the electron mediator and, optionally, the electrocatalyst, and which is capable of immobilizing the organelle. In various embodiments, the organelle immobilization material is further capable of stabilizing the organelle.

In an alternative embodiment of the biocathode, there is no electrocatalyst present. In this embodiment, the electrons combine with an oxidized form of the electron mediator to produce a reduced form of the electron mediator. Then, the reduced form of the electron mediator reacts with an oxidized form of an oxidant to produce an oxidized form of the electron mediator and water. In one embodiment, an organelle immobilization material permeable to the oxidant is present, which optionally comprises the electron mediator, and which is capable of immobilizing the enzyme. In some of the embodiments, the organelle immobilization material is further capable of stabilizing the organelle. Further, in various embodiments, a biocathode comprising an electron conductor, an organelle, and an oxidant is used wherein an enzyme contained within the organelle is capable of accepting electrons from the electron conductor.

The biofuel cell of the present invention comprises a biocathode and/or a bioanode. Generally, the bioanode comprises elements that effect the oxidation of fuel fluid whereby electrons are released and directed to an external electrical load. The resulting electrical current powers the electrical load, with electrons being subsequently directed to a biocathode where an oxidant is reduced and water is produced.

For the bioelectrodes described above, in various embodiments, at least two organelles are used. When at least two organelles are used, in preferred embodiments, the organelles used are thylakoid and mitochondria or mitoplasts.

A. Bioanode

The bioanode in accordance with this invention comprises an electron conductor, and an organelle that is immobilized in an organelle immobilization material. In one embodiment, these components are adjacent to one another, meaning they are physically or chemically connected by appropriate means.

1. Electron Conductor

The electron conductor is a substance that conducts electrons. The electron conductor can be organic or inorganic in nature as long as it is able to conduct electrons through the material. The electron conductor can be a carbon-based material, stainless steel, stainless steel mesh, a metallic conductor, a semiconductor, a metal oxide, a modified conductor, or combinations thereof. In preferred embodiments, the electron conductor is a carbon-based material.

Particularly suitable electron conductors are carbon-based materials. Exemplary carbon-based materials are carbon cloth, carbon paper, carbon screen printed electrodes, carbon paper (Toray), carbon paper (ELAT), carbon black (Vulcan XC-72, E-tek), carbon black, carbon powder, carbon fiber, single-walled carbon nanotubes, double-walled carbon nanotubes, multi-walled carbon nanotubes, carbon nanotubes arrays, diamond-coated conductors, glassy carbon, mesoporous carbon, and combinations thereof. In addition, other exemplary carbon-based materials are graphite, uncompressed graphite worms, delaminated purified flake graphite (Superior® graphite), high performance graphite and carbon powders (Formula BT™, Superior® graphite), highly ordered pyrolytic graphite, pyrolytic graphite, polycrystalline graphite, and combinations thereof. A preferred electron conductor (support membrane) is a sheet of carbon paper.

In a further embodiment, the electron conductor can be made of a metallic conductor. Suitable electron conductors can be prepared from gold, platinum, iron, nickel, copper, silver, stainless steel, mercury, tungsten, other metals suitable for electrode construction, and combinations thereof. In addition, electron conductors which are metallic conductors can be constructed of nanoparticles made of cobalt, carbon, and other suitable metals. Other metallic electron conductors can be silver-plated nickel screen printed electrodes.

In addition, the electron conductor can be a semiconductor. Suitable semiconductor materials include silicon and germanium, which can be doped with other elements. The semiconductors can be doped with phosphorus, boron, gallium, arsenic, indium or antimony, or a combination thereof.

Other electron conductors can be metal oxides, metal sulfides, main group compounds (i.e., transition metal compounds), materials modified with electron conductors, and combinations thereof Exemplary electron conductors of this type are nanoporous titanium oxide, tin oxide coated glass, indium tin oxide coated glass, cerium oxide particles, molybdenum sulfide, boron nitride nanotubes, aerogels modified with a conductive material such as carbon, solgels modified with conductive material such as carbon, ruthenium carbon aerogels, mesoporous silicas modified with a conductive material such as carbon, conductive polymers, composites of conducting polymers and other electron conductors (e.g., composite of conducting polymers and carbon nanotubes), and combinations thereof.

In various preferred embodiments, the electron conductor is a carbon cloth, a carbon nanotube, an expanded graphite worm, a carbon paste, and combinations thereof More preferably, the electron conductor is a carbon nanotube.

2. Electron Mediators

The electron mediators can be added in addition to the immobilized organelle or they can be contained entirely within the immobilized organelle. The bioanode electron mediator serves to accept or donate electron(s), readily changing from oxidized to reduced forms. The electron mediator is a compound that can diffuse into the immobilization material and/or be incorporated into the immobilization material. It is preferred that the electron mediator's diffusion coefficient is maximized.

Exemplary electron mediators are nicotinamide adenine dinucleotide (NAD⁺), flavin adenine dinucleotide (FAD), nicotinamide adenine dinucleotide phosphate (NADP), pyrroloquinoline quinone (PQQ), equivalents of each, and combinations thereof. Other exemplary electron mediators are phenazine methosulfate, dichlorophenol indophenol, short chain ubiquinones, potassium ferricyanide, a protein, a metalloprotein, stellacyanin, and combinations thereof. In various embodiments, the organelle comprises a protein or metalloprotein that can act as an electron mediator.

Where the electron mediator cannot undergo a redox reaction at the electron conductor by itself, the bioanode comprises an electrocatalyst for an electron mediator which facilitates the release of electrons at the electron conductor. Alternatively, a reversible redox couple that has a standard reduction potential of 0.0V±0.5 V is used as the electron mediator. Stated another way, an electron mediator that provides reversible electrochemistry on the electron conductor surface can be used. The electron mediator is coupled with a naturally occurring enzyme contained in an organelle that is dependent on that electron mediator.

3. Electrocatalyst for an Electron Mediator

Generally, the electrocatalyst is a substance that facilitates the release of electrons at the electron conductor. Stated another way, the electrocatalyst improves the kinetics of a reduction or oxidation of an electron mediator so the electron mediator reduction or oxidation can occur at a lower standard reduction potential. The electrocatalyst can be reversibly oxidized at the bioanode to produce electrons and thus, electricity. When the electrocatalyst is adjacent to the electron conductor, the electrocatalyst and electron conductor are in electrical contact with each other, but not necessarily in physical contact with each other. In one embodiment, the electron conductor is part of, associates with, or is adjacent to an electrocatalyst for an electron mediator.

Generally, the electrocatalyst can be an azine, a conducting polymer or an electroactive polymer. Exemplary electrocatalysts are methylene green, methylene blue, luminol, nitro-fluorenone derivatives, azines, osmium phenanthrolinedione, catechol-pendant terpyridine, toluene blue, cresyl blue, nile blue, neutral red, phenazine derivatives, tionin, azure A, azure B, azure C, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), poly(methylene green), poly(methylene blue), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b′]dithiophene), poly(3-(4-fluorophenyl)thiophene), poly(neutral red), a protein, a metalloprotein, stellacyanin, and combinations thereof In one preferred embodiment, the electrocatalyst for the electron mediator is poly(methylene green). In various embodiments, the organelle comprises a protein or metalloprotein that can act as an electrocatalyst for an electron mediator.

4. Organelle

An enzyme or group of enzymes in an organelle catalyzes the oxidation of the fuel fluid at the bioanode. Any organelle that contains enzymes and/or enzymes and electron mediators capable of oxidizing a fuel fluid can be used as the anode organelle of the invention. Specifically, glyoxysome, peroxisome, mitochondria, mitoplasts, and combinations thereof can be immobilized and used in the bioanode. In various preferred embodiments, the organelle is mitochondria or mitoplasts.

The organelles contain various enzymes. For example, mitochondria or mitoplasts contain the enzymes necessary for the citric acid cycle that oxidize pyruvate produced by glycolysis to ATP, NADH, FADH₂ and CO₂. Mitoplasts are mitochondria with the outer membrane removed. Generally, these mitochondrial enzymes are aconitase, fumarase, malate dehydrogenase, succinate dehydrogenase, succinyl-CoA synthetase, isocitrate dehydrogenase, ketoglutarate dehydrogenase, and citrate synthase. Mitochondria and mitoplasts contain the enzymes and coenzymes of the Kreb's cycle and the electron transport chain, so they are ideally designed for completely oxidizing common fuel fluids, but unlike a microbe, they have fewer transport limitations due to smaller diffusion lengths, no biofilm formation, and no need to transport fuel across the cell wall; these differences lead to higher power densities.

The enzymes contained in glyoxysome include malate synthase, malate dehydrogenase, citrate (Si)-synthase, aconitate hydratase, and isocitrate lyase. Further the enzymes contained in peroxisomes include catalase, D-amino acid oxidase, and uric acid oxidase.

Organelles, particularly mitochondria, can be more difficult to immobilize than isolated enzymes. The organelles require a particular range of osmotic pressures that mimic those in the cell, so the organelle will not either expand or contract to and extent that damages the organelle. Also, because the organelle used typically has a membrane, the mass transport of the fuel fluid to the enzyme(s) that catalyze the oxidation is maximized, so the current density of the biofuel cell does not decrease.

In some of the various embodiments, the organelles are isolated from the cell. Stated another way, these organelles are extracted from the cell and contain the contents of the specific organelle surrounded by its particular membrane only. For example, when the immobilized organelle is mitochondria, the distinct mitochondria organelle with its two membranes is immobilized within the immobilization material. These organelles are not contained within or immobilized within a cell.

In various other embodiments, rather than an immobilized organelle, the bioanode as described in this bioanode section comprises various enzymes. In some of these embodiments, the enzymes are the enzymes of the Krebs cycle. For example, aconitase, fumarase, malate dehydrogenase, succinate dehydrogenase, succinyl-CoA synthetase, isocitrate dehydrogenase, ketoglutarate dehydrogenase, citrate synthase and combinations thereof can be immobilized in immobilization materials described herein to prepare a bioanode. By way of further example, the glycolysis cycle enzymes can also be immobilized in a bioanode. For example, hexokinase (HK), phosphoglucose isomerase, phosphofructose kinase (PFK-1), pyrophosphate-dependent phosphofructokinase (PFP), aldolase (ALDO), triosephosphate isomerase (TPI), glyceraldehyde-3-phosphate dehydrogenase (GAPDH), phosphoglycerate kinase (PGK), phosphoglycerate mutase (PGM), enolase (ENO), pyruvate kinase (PK), and combinations thereof can also be immobilized in immobilization materials described herein to prepare a bioanode.

5. Organelle Immobilization Material

An organelle immobilization material is utilized in the biofuel cell at the bioanode and/or the biocathode. The bioanode's organelle immobilization material is permeable to the fuel fluid and immobilizes the organelle. The immobilization material is permeable to the fuel fluid so the oxidation reaction of the fuel at the bioanode can be catalyzed by the enzyme(s) contained within the immobilized organelle. In some embodiments, the immobilization material is further capable of stabilizing the organelle.

Generally, an enzyme or group of enzymes contained within an organelle is used to catalyze redox reactions at the biocathode and/or the bioanode. In a bioanode and/or biocathode according to this invention, an organelle is immobilized in an organelle immobilization material that immobilizes the organelle. In some cases, the organelle immobilization material further stabilizes the organelle. Typically, a free enzyme in solution loses its catalytic activity within a few hours to a few days, whereas an enzyme within a properly immobilized organelle can retain its catalytic activity for at least about 7 days to about 730 days. The retention of catalytic activity is defined as the enzyme having at least about 75% of its initial activity, which can be measured by chemiluminescence, electrochemical, UV-Vis, radiochemical, or fluorescence assay. The enzyme retains at least about 75% of its initial activity while the biofuel cell is continually producing electricity for at least about 7 days to about 730 days.

An immobilized organelle is an organelle that is physically confined in a certain region of the organelle immobilization material while retaining its activity. There are a variety of methods for organelle immobilization, including carrier-binding, cross-linking and entrapping. Carrier-binding is the binding of organelles to water-insoluble carriers. Cross-linking is the intermolecular cross-linking of organelles by bifunctional or multifunctional reagents. Entrapping is incorporating organelles into the lattices of a semipermeable material. The particular method of organelle immobilization is not critically important, so long as the organelle immobilization material (1) immobilizes the organelle, (2) stabilizes the organelle, and (3) is permeable to the fuel fluid or oxidant.

With reference to the organelle immobilization material's permeability to the fuel fluid or oxidant and the immobilization of the organelle, in various embodiments, the material is permeable to a compound that is smaller than an organelle. Stated another way, the organelle immobilization material allows the movement of the fuel fluid or oxidant compound through it so the compound can contact the enzyme(s) within the organelle. The organelle immobilization material can be prepared in a manner such that it contains internal pores, channels, openings or a combination thereof, which allow the movement of the compound throughout the organelle immobilization material, but which provide a chemical environment conducive to organelle activity. Such a chemical environment allows the organelle to retain its catalytic activity. In various preferred embodiments, the organelle is provided a chemical environment that is hydrophobic, buffered near a neutral pH, at a temperature from about 0 to about 40° C., and provides sufficient nutrients to the organelle.

In various embodiments, the organelle is preferably located within the organelle immobilization material and the compound travels in and out of the organelle immobilization material through transport channels. The organelle immobilization material typically also has pores. Further, a transport channel preferably has a diameter of at least about 10 nm. In still another embodiment, the pore diameter to transport channel diameter ratio is at least about 2:1, 2.5:1, 3:1, 3.5:1, 4:1, 4.5:1, 5:1, 5.5:1, 6: 1, 6.5:1, 7:1, 7.5:1, 8:1, 8.5:1, 9:1, 9.5:1, 10:1 or more. In yet another embodiment, preferably, a transport channel has a diameter of at least about 10 nm and the pore diameter to transport channel diameter ratio is at least about 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1 or more.

With respect to the stabilization of the organelle, the organelle immobilization material provides a chemical and mechanical barrier to prevent or impede the organelle's destruction. To this end, the organelle is found within the immobilization material. Also, the organelle immobilization material provides a chemical barrier to maximize the organelle's activity upon immobilization. For example, a preferred chemical environment for immobilized mitochondria is hydrophobic, buffered near a neutral pH, at a temperature from about 0 to about 40° C., and includes sufficient nutrients for mitochondria activity.

In some of the embodiments, the organelle immobilization material has a micellar or inverted micellar structure. Generally, the molecules making up a micelle are amphipathic, meaning they contain a polar, hydrophilic group and a nonpolar, hydrophobic group. The molecules can aggregate to form a micelle, where the polar groups are on the surface of the aggregate and the hydrocarbon, nonpolar groups are sequestered inside the aggregate. Inverted micelles have the opposite orientation of polar groups and nonpolar groups. The amphipathic molecules making up the aggregate can be arranged in a variety of ways so long as the polar groups are in proximity to each other and the nonpolar groups are in proximity to each other. Also, the molecules can form a bilayer with the nonpolar groups pointing toward each other and the polar groups pointing away from each other. Alternatively, a bilayer can form wherein the polar groups can point toward each other in the bilayer, while the nonpolar groups point away from each other.

Generally, the micellar or inverted micellar organelle immobilization material can be a polymer, a ceramic, a liposome, or any other material made of molecules that form a micellar or inverted micellar structure. Exemplary micellar or inverted micellar organelle immobilization materials are perfluoro sulfonic acid-polytetrafluoro ethylene (PTFE) copolymer (or perfluorinated ion exchange polymer)(Nafion® or Flemion®), modified perfluoro sulfonic acid-polytetrafluoro ethylene (PTFE) copolymer (or modified perfluorinated ion exchange polymer)(modified Nafion® or modified Flemion®), polysulfone, micellar polymers, poly(ethylene oxide) based block copolymers, polymers formed from microemulsion and/or micellar polymerization and copolymers of alkyl methacrylates, alkyl acrylates, and styrenes. Other exemplary micellar or inverted micellar immobilization materials are ceramics, sodium bis(2-ethylhexyl)sulfosuccinate, sodium dioctylsulfosuccinate, lipids, phospholipids, sodium dodecyl sulfate, decyltrimethylammonium bromide, tetradecyltrimethylammonium bromide, (4-[(2-hydroxyl-1-naphthalenyl)azo]benzenesulfonic acid monosodium salt), linoleic acids, linolenic acids, colloids, liposomes and micelle networks.

In one preferred embodiment, the micellar organelle immobilization material is a modified perfluoro sulfonic acid-PTFE copolymer (or modified perfluorinated ion exchange polymer)(modified Nafion® or modified Flemion®) membrane. The perfluorinated ion exchange polymer membrane is modified with a hydrophobic cation that is larger than the ammonium (NH₄⁻) ion. The hydrophobic cation serves the dual function of (1) dictating the membrane's pore size and (2) acting as a chemical buffer to help maintain the pore's pH level, both of which further efforts to stabilize the organelle.

With regard to the first function of the hydrophobic cation, mixture-casting a perfluoro sulfonic acid-PTFE copolymer (or perfluorinated ion exchange polymer) with a hydrophobic cation to produce a modified perfluoro sulfonic acid-PTFE copolymer (or modified perfluorinated ion exchange polymer)(Nafion® or Flemion®) membrane provides an organelle immobilization material wherein the pore size is dependent on the size of the hydrophobic cation. Accordingly, the larger the hydrophobic cation, the larger the pore size. This function of the hydrophobic cation allows the pore size to be made larger or smaller to fit a specific organelle by varying the size of the hydrophobic cation.

Regarding the second function of the hydrophobic cation, the properties of the perfluoro sulfonic acid-PTFE copolymer (or perfluorinated ion exchange polymer) membrane are altered by exchanging the hydrophobic cation for protons as the counterion to the —SO₃⁻ groups on the perfluoro sulfonic acid-PTFE copolymer (or perfluorinated ion exchange polymer) membrane. This change in counterion provides a buffering effect on the pH because the hydrophobic cation has a much greater affinity for the −SO₃⁻ sites than protons do. This buffering effect of the membrane causes the pH of the pore to remain substantially unchanged with changing solution pH; stated another way, the pH of the pore resists changes in the solution's pH. In addition, the membrane provides a mechanical barrier, which further protects the immobilized organelles.

Table 1 demonstrates the buffering effect of the modified perfluoro sulfonic acid-PTFE copolymer membrane. The values represent the number of available exchange sites for protons per gram of modified perfluoro sulfonic acid-PTFE copolymer membrane; as the number of exchange sites available to protons decreases, the buffering capacity of the membrane toward the immobilized organelle increases. The membrane abbreviations designate the following membranes: NH₄Br is an ammonium bromide-modified Nafion® membrane, TMABr is a tetramethylammonium bromide-modified Nafion® membrane, TEABr is a tetraethylammonium bromide-modified Nafion® membrane, TpropABr is a tetrapropylammonium bromide-modified Nafion® membrane, TBABr is a tetrabutylammonium bromide-modified Nafion® membrane, and TpentABr is a tetrapentylammonium bromide-modified Nafion® membrane.

TABLE 1

| Membrane | Mixture-Cast (×10⁻⁶ mole/g) | Salt-Extracted (×10⁻⁶ mole/g) |
|---|---|---|
| Nafion ® | 907 ± 68 | — |
| NH₄Br | 521 ± 74 | 591 ± 95 |
| TMABr | 171 ± 19 | 458 ± 27 |
| TEABr | 157 ± 4 | 185 ± 22 |
| TPropABr | 133 ± 6 | 138 ± 77 |
| TBABr | 8.68 ± 2.12 | 96 ± 23 |
| TPentABr | 2.71 ± 0.6 | 1.78 ± 1.66 |

In order to prepare a modified perfluoro sulfonic acid-PTFE copolymer (or perfluorinated ion exchange polymer) membrane, the first step is to cast a suspension of perfluoro sulfonic acid-PTFE copolymer (or perfluorinated ion exchange polymer), particularly Nafion®, with a solution of the hydrophobic cations to form a membrane. After extracting the excess hydrophobic cations and their salts from the original membrane, the membrane is re-cast. Upon re-casting, the membrane contains the hydrophobic cations in association with the —SO₃⁻ sites of the perfluoro sulfonic acid-PTFE copolymer (or perfluorinated ion exchange polymer) membrane.

In order to make more stable and reproducible quaternary ammonium salt-treated Nafion® membranes, the excess bromide salts must be removed from the casting solution. This salt-extracted membrane is formed by re-casting the mixture-cast membranes after the excess quaternary ammonium bromide and HBr salts have been extracted from the original membranes. Salt extraction of membranes retains the presence of the quaternary ammonium cations at the sulfonic acid exchange sites, but eliminates complications from excess salt that may be trapped in the pore or may cause voids in the equilibrated membrane. The chemical and physical properties of the salt-extracted membranes have been characterized by voltammetry, ion exchange capacity measurements, and fluorescence microscopy before organelle immobilization. Exemplary hydrophobic cations are ammonium-based cations, quaternary ammonium cations, alkyltrimethylammonium cations, alkyltriethylammonium cations, organic cations, phosphonium cations, triphenylphosphonium, pyridinium cations, imidazolium cations, hexdecylpyridinium, ethidium, viologens, methyl viologen, benzyl viologen, bis(triphenylphosphine)iminium, metal complexes, bipyridyl metal complexes, phenanthroline-based metal complexes, [Ru(bipyridine)₃]²⁺, [Fe(phenanthroline)₃]³⁺, and combinations thereof.

In one preferred embodiment, the hydrophobic cations are ammonium-based cations. In particular, the hydrophobic cations are quaternary ammonium cations. In another embodiment, the quaternary ammonium cations are represented by formula (1):

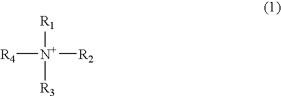

wherein R₁, R₂, R₃, and R₄ are independently hydrogen, hydrocarbyl, substituted hydrocarbyl, or heterocyclo wherein at least one of R₁, R₂, R₃, and R₄ is other than hydrogen. In a further embodiment, preferably, R₁, R₂, R₃, and R₄ are independently hydrogen, methyl, ethyl, propyl, butyl, pentyl, hexyl, heptyl, octyl, nonyl, decyl, undecyl, dodecyl, tridecyl or tetradecyl wherein at least one of R₁, R₂, R₃, and R₄ is other than hydrogen. In still another embodiment, R₁, R₂, R₃, and R₄ are the same and are methyl, ethyl, propyl, butyl, pentyl or hexyl. In yet another embodiment, preferably, R₁, R₂, R₃, and R₄ are butyl. Alternatively, the quaternary ammonium cation is hexyltriethylammonium, octyltrimethylammonium, decyltrimethylammonium, dodecyl trimethylammonium, tetradecyltrimethylammonium, hexadecyltrimethylammonium, or octadecyltrimethylammonium.

Mixture-cast films of quaternary ammonium salts or surfactants (e.g., TBAB, triethylhexylammonium bromide, trimethyldodecylammonium bromide, and phenyltrimethylammonium bromide) and Nafion® have increased the mass transport of small analytes through the films and decreased the selectivity of the organelle immobilization membrane against anions. These organelle immobilization membranes have very similar conductivities as unmodified Nafion®, but they have a much higher preference to the quaternary ammonium bromide than to the proton, as shown by titrating the number of available exchange sites to protons in the organelle immobilization membranes. Therefore, these films have similar electrical properties, but very different acid/base properties. The treated organelle immobilization membranes maintain their neutral pH over a wide range of buffer pHs. In light of these advantages, the preferred organelle immobilization material is a quaternary ammonium salt treated perfluoro sulfonic acid-PTFE copolymer (or modified perfluorinated ion exchange polymer)(modified Nafion® or modified Flemion®) membrane. More preferably, the organelle immobilization material is a TBAB-modified Nafion® membrane material.

The organelle immobilization materials described above can also be used to immobilize enzymes as described in U.S. patent application Ser. No. 10/931,147 (published as U.S. Patent Application Publication No. 2005/0095466), and U.S. patent application Ser. No. 10/617,452 (published as U.S. Patent Application Publication No. 2004/0101741). When these immobilization materials are used to immobilize enzymes, various modified-perfluoro sulfonic acid-PTFE copolymers are particularly suited to immobilize certain enzymes. For example, for the glycolysis cycle enzymes of hexokinase and phosphofructose kinase, immobilization in trimethyldodecylammonium modified Nafion® is advantageous. For the glycolysis cycle enzymes of phosphoglucose isomerase and glyceraldehyde-3-phosphate dehydrogenase, immobilization in tetrabutylammonium modified Nafion® is advantageous and for the glycolysis cycle enzyme of aldolase, immobilization in trimethyltetradecylammonium modified Nafion® is advantageous.

Certain enzyme immobilization materials, and particularly micellar enzyme immobilization materials and modified-perfluoro sulfonic acid-PTFE copolymers, are described in U.S. patent application Ser. No. 10/931,147 (published as U.S. Patent Application Publication No. 2005/0095466), and U.S. patent application Ser. No. 10/617,452 (published as U.S. Patent Application Publication No. 2004/0101741), both of which are herein incorporated by reference in their entirety. These enzyme immobilization materials are suitable organelle immobilization materials for purposes of the present invention.

Exemplary micellar or inverted micellar organelle immobilization materials are polycationic polymers, such as hydrophobically modified polysaccharides, particularly, hydrophobically modified chitosan. In various embodiments, the micellar hydrophobically modified chitosan corresponds to the structure of Formula 2

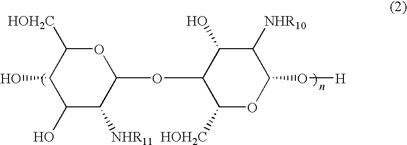

wherein n is an integer; R₁₀ is independently hydrogen, hydrocarbyl, substituted hydrocarbyl, or a hydrophobic redox mediator; and R₁₁ is independently hydrogen, hydrocarbyl, substituted hydrocarbyl, or a hydrophobic redox mediator. In certain embodiments of the invention, n is an integer that gives the polymer a molecular weight of from about 21,000 to about 500,000; preferably, from about 90,000 to about 500,000; more preferably, from about 150,000 to about 350,000; more preferably, from about 225,000 to about 275,000. In many embodiments, R₁₀ is independently hydrogen or alkyl and R₁₁ is independently hydrogen or alkyl. Further, R₁₀ is independently hydrogen or hexyl and R₁₁ is independently hydrogen or hexyl. Alternatively, R₁₀ is independently hydrogen or octyl and R₁₁ is independently hydrogen or octyl.

In other various embodiments, the micellar hydrophobically modified chitosan is a micellar hydrophobic redox mediator modified chitosan corresponding to Formula 3

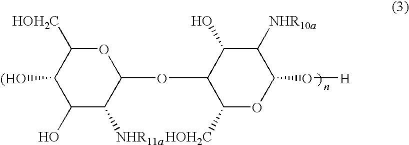

wherein n is an integer; R₁₀ₐ is independently hydrogen, or a hydrophobic redox mediator; and R₁₁ₐ is independently hydrogen, or a hydrophobic redox mediator.

The micellar hydrophobically modified chitosans can be modified with hydrophobic groups to varying degrees. The degree of hydrophobic modification is determined by the percentage of free amine groups that are modified with hydrophobic groups as compared to the number of free amine groups in the unmodified chitosan. The degree of hydrophobic modification can be estimated from an acid-base titration and/or nuclear magnetic resonance (NMR), particularly ¹H NMR, data. This degree of hydrophobic modification can vary widely and is at least about 1, 2, 4, 6, 8, 10, 12, 14, 16, 18, 20, 25, 30, 32, 24, 26, 28, 40, 42, 44, 46, 48%, or more. Preferably, the degree of hydrophobic modification is from about 10% to about 45%; from about 10% to about 35%; from about 20% to about 35%; or from about 30% to about 35%.

In other various embodiments, the hydrophobic redox mediator of Formula 3is a transition metal complex of osmium, ruthenium, iron, nickel, rhodium, rhenium, or cobalt with 1,10-phenanthroline (phen), 2,2′-bipyridine (bpy) or 2,2′,2″-terpyridine (terpy), methylene green, methylene blue, poly(methylene green), poly(methylene blue), luminol, nitro-fluorenone derivatives, azines, osmium phenanthrolinedione, catechol-pendant terpyridine, toluene blue, cresyl blue, nile blue, neutral red, phenazine derivatives, tionin, azure A, azure B, azure C, toluidine blue O, acetophenone, metallophthalocyanines, nile blue A, modified transition metal ligands, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), quinones, diimines, diaminobenzenes, diaminopyridines, phenothiazine, phenoxazine, toluidine blue, brilliant cresyl blue, 3,4-dihydroxybenzaldehyde, poly(acrylic acid), poly(azure I), poly(nile blue A), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b′]dithiophene), poly(3-(4-fluorophenyl)thiophene), poly(neutral red), or combinations thereof.

Preferably, the hydrophobic redox mediator is Ru(phen)₃⁺², Fe(phen)₃⁺², Os(phen)₃⁺², Co(phen)₃⁺², Cr(phen)₃⁺², Ru(bpy)₃⁺², Os(bpy)₃⁺², Fe(bpy)₃⁻², Co(bpy)₃⁻², Cr(bpy)₃⁺², Os(terpy)₃⁺², Ru(bpy)₂(4-methyl-4′-(6-hexyl)-2,2′-bipyridine)⁻², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine), Os(bpy)₂(4-methyl-4′-(6-hexyl)-2,2′-bipyridine)⁺², or combinations thereof. More preferably, the hydrophobic redox mediator is Ru(bpy)₂(4-methyl-4′-(6-hexyl)-2,2′-bipyridine)⁺², Co(bpy)₂(4-methyl-4′-(6-hexyl)-2,2′-bipyridine)⁺², Cr(bpy)₂(4-methyl-4′-(6-hexyl)-2,2′-bipyridine)⁺², Fe(bpy)₂(4-methyl-4′-(6-hexyl)-2,2′-bipyridine)⁺², Os(bpy)₂(4-methyl-4′-(6-hexyl)-2,2′-bipyridine)⁺², or combinations thereof. In various preferred embodiments, the hydrophobic redox mediator is Ru(bpy)₂(4-methyl-4′-(6-hexyl)-2,2′-bipyridine)⁺².

For the immobilization material having a hydrophobic redox mediator as the modifier, the hydrophobic redox mediator is typically covalently bonded to the chitosan or polysaccharide backbone. Typically, in the case of chitosan, the hydrophobic redox mediator is covalently bonded to one of the amine functionalities of the chitosan through a —N—C— bond. In the case of metal complex redox mediators, the metal complex is attached to the chitosan through an —N—C— bond from a chitosan amine group to an alkyl group attached to one or more of the ligands of the metal complex. A structure corresponding to Formula 4 is an example of a metal complex attached to a chitosan

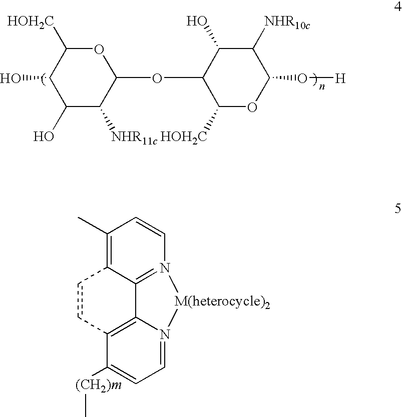

wherein n is an integer; R_{10c} is independently hydrogen or a structure corresponding to Formula 5; R_{11c} is independently hydrogen or a structure corresponding to Formula 5; m is an integer from 0 to 10; M is Ru, Os, Fe, Cr, or Co; and heterocycle is bipyridyl, substituted bipyridyl, phenanthroline, acetylacetone, and combinations thereof.

The hydrophobic group used to modify chitosan serves the dual function of (1) dictating the immobilization material's pore size and (2) modifying the chitosan's electronic environment to maintain an acceptable pore environment, both of which stabilize the enzyme. With regard to the first function of the hydrophobic group, hydrophobically modifying chitosan produces an organelle immobilization material wherein the pore size is dependent on the size of the hydrophobic group. Accordingly, the size, shape, and extent of the modification of the chitosan with the hydrophobic group affects the size and shape of the pore. This function of the hydrophobic cation allows the pore size to be made larger or smaller or a different shape to fit a specific enzyme by varying the size and branching of the hydrophobic group.

Regarding the second function of the hydrophobic cation, the properties of the hydrophobically modified chitosan membranes are altered by modifying chitosan with hydrophobic groups. This hydrophobic modification of chitosan affects the pore environment by increasing the number of available exchange sites to proton. In addition to affecting the pH of the material, the hydrophobic modification of chitosan provides a membrane that is a mechanical barrier, which further protects the immobilized enzymes.

Table 2 shows the number of available exchange sites to proton for the hydrophobically modified chitosan membrane.

TABLE 2
Number of available exchange sites to proton per gram
of chitosan polymer

| | Exchange sites per gram |
|---|---|
| Membrane | (×10⁻⁴ mol SO₃/g) |
| Chitosan | 10.5 ± 0.8 |
| Butyl Modified | 226 ± 21 |
| Hexyl Modified | 167 ± 45 |
| Octyl Modified | 529 ± 127 |
| Decyl Modified | 483 ± 110 |

To prepare the hydrophobically modified chitosans of the invention having an alkyl group as a modifier, a chitosan gel was suspended in acetic acid followed by addition of an alcohol solvent. To this chitosan gel was added an aldehyde (e.g., butanal, hexanal, octanal, or decanal), followed by addition of sodium cyanoborohydride. The resulting product was separated by vacuum filtration and washed with an alcohol solvent. The modified chitosan was then dried in a vacuum oven at 40° C., resulting in a flaky white solid.

To prepare a hydrophobically modified chitosan of the invention having a redox mediator as a modifier, a redox mediator ligand was derivatized by contacting 4,4′-dimethyl-2,2′-bipyridine with lithium diisopropylamine followed by addition of a dihaloalkane to produce 4-methyl-4′-(6-haloalkyl)-2,2′-bipyridine. This ligand was then contacted with Ru(bipyridine)₂Cl₂ hydrate in the presence of an inorganic base and refluxed in a water-alcohol mixture until the Ru(bipyridine)₂Cl₂ was depleted. The product was then precipitated with ammonium hexafluorophosphate, or optionally a sodium or potassium perchlorate salt, followed by recrystallization. The derivatized redox mediator (Ru(bipyridine)₂(4-methyl-4′-(6-bromohexyl)-2,2′-bipyridine)⁻²) was then contacted with deacetylated chitosan and heated. The redox mediator modified chitosan was then precipitated and recrystallized.

In various embodiments, the organelle immobilization material is non-microbial. In these embodiments, a non-microbial organelle immobilization material is an organelle immobilization material that is not excreted by microbes. Further, the organelle immobilization material is not a biofilm that is excreted by microbes.

6. Bioanode Embodiments

In addition to the organelle containing bioanodes, a preferred bioanode for use in biofuel cells of the invention is described in U.S. patent application Ser. No. 10/617,452 (published as U.S. Patent Application Publication No. 2004/0101741), which is incorporated herein by reference in its entirety. Other potentially useful bioanodes are described in U.S. Pat. Nos. 6,531,239 and 6,294,281, which are also incorporated herein by reference.

B. Biocathode

In one embodiment, the biocathode comprises an electron conductor and an organelle which is immobilized in an organelle immobilization material. The above-identified components of the biocathode are adjacent to one another; meaning they are physically or chemically connected by appropriate means. As the components are generally the same as the bioanode components, the following discussion concerns the differences in composition of the respective elements and differences in function, where appropriate.

1. Electron Conductor

As with the bioanode, the biocathode's electron conductor can be organic or inorganic in nature as long as it is able to conduct electrons through the material. In one embodiment, the biocathode electron conductor is carbon cloth.

2. Electron Mediator

The biocathode's electron mediator can be included external to the biocathode's organelle. In various embodiments, an electron mediator is included in the biocathode in addition to the electron mediator within the organelle. The electron mediators can be a protein such as stellacyanin, a protein byproduct such as bilirubin, a sugar such as glucose, a sterol such as cholesterol, a fatty acid, ferrodoxin, or a metalloprotein. The electron mediators can also be a coenzyme or substrate of an oxidase.

3. Electrocatalyst for an Electron Mediator

Generally, the electrocatalyst is a substance that facilitates the release of electrons at the electron conductor by reducing the standard reduction potential of the electron mediator.

Typically, electrocatalysts according to the invention are organometallic cations with standard reduction potentials greater than +0.4 volts. Exemplary electrocatalysts are transition metal complexes, such as osmium, ruthenium, iron, nickel, rhodium, rhenium, and cobalt complexes. Preferred organometallic cations using these complexes comprise large organic aromatic ligands that allow for large electron self exchange rates. Examples of large organic aromatic ligands include derivatives of 1,10-phenanthroline (phen), 2,2′-bipyridine (bpy) and 2,2′,2″-terpyridines (terpy), such as Ru(phen)₃⁺², Fe(phen)₃⁻², Ru(bpy)₃⁺², Os(bpy)₃⁺², and Os(terpy)₃⁺². In a preferred embodiment, the electrocatalyst is a ruthenium compound. Most preferably, the electrocatalyst at the biocathode is Ru(bpy)₃⁻² (represented by Formula 6).

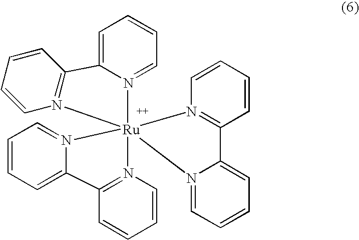

The electrocatalyst is present in a concentration that facilitates the efficient transfer of electrons. Preferably, the electrocatalyst is present at a concentration that makes the organelle immobilization material conduct electrons. Particularly, the electrocatalyst is present at a concentration of from about 10 mM to about 3 M, more preferably from about 250 mM to about 2.25 M, still more preferably from about 500 mM to about 2 M, and most preferably from about 1.0 M to about 1.5 M.

4. Organelle

In accordance with the invention, an enzyme or group of enzymes in an organelle reduces an oxidant at the biocathode. Any organelle that contains enzymes and/or enzymes and electron mediators capable of reducing an oxidant can be used as the cathode organelle. Specifically, thylakoids, chloroplasts, hydrogenosomes, or combinations thereof can be immobilized in the biocathode. In preferred embodiments, thylakoids or chloroplasts are immobilized in the biocathode.

Generally, the enzymes contained within the chloroplast include RuBisCO, phosphoglycerate kinase, G3P dehydrogenase, triose phosphate isomerase, aldolase, fructose-1,6-bisphosphatase, transketolase, S1,7BPase, epimerase, ribose isomerase, and phosphoribulokinase. The enzymes within the hydrogenosome include succinyl-CoA synthetase.

5. Organelle Immobilization Material

As described above, an organelle immobilization material is utilized in the biofuel cell at the bioanode and/or the biocathode. Further detail regarding the composition of the organelle immobilization material and the immobilization mechanism can be found above at I.A.5. In one embodiment, the biocathode's organelle immobilization material is permeable to the oxidant and immobilizes the organelle. In some embodiments, the immobilization material further stabilizes the organelle. The immobilization material is permeable to the oxidant so the reduction of the oxidant at the biocathode can be catalyzed by the immobilized organelle.

6. Biocathode Embodiments

In addition to the organelle-containing biocathodes, various biocathodes can be incorporated into the biofuel cells of the present invention. For example, biocathodes are described in U.S. patent application Ser. No. 10/931,147 (published as U.S. Patent Application Publication No. 2005/0095466), herein incorporated by reference in its entirety.

C. Fuel Fluid and Oxidant

A fuel fluid that can be oxidized to produce electrons at the bioanode and an oxidant that can be reduced to produce water at the biocathode are components of the biofuel cell of this invention.

The fuel fluid for the bioanode is consumed in the oxidation reaction of a redox center of the enzyme(s) contained in the immobilized organelle. The fuel fluid's molecular size is small enough so the diffusion coefficient through the organelle immobilization material is large. Exemplary fuel fluids are hydrogen, ammonia, alcohols (such as methanol, ethanol, propanol, isobutanol, butanol and isopropanol), allyl alcohols, aryl alcohols, glycerol, propanediol, mannitol, glucuronate, aldehyde, carbohydrates (such as glucose, glucose-1, D-glucose, L-glucose, glucose-6-phosphate, lactate, lactate-6-phosphate, D-lactate, L-lactate, fructose, galactose-1, galactose, aldose, sorbose and mannose), glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acyl-CoA, steroids, amino acids, flavin, NADH, NADH₂, NADPH, NADPH₂, hydrocarbons, amines, ATP, and combinations thereof. In various preferred embodiments, when the organelle is mitochondria, the fuel fluid is glucose, pyruvate, and combinations thereof.

The oxidant for the biocathode is consumed in the reduction reaction of a redox center of the immobilized organelle using electrons supplied by the bioanode. The oxidant's molecular size is small enough so the diffusion coefficient through the organelle immobilization material is large. A variety of means of supplying a source of the oxidant known in the art can be utilized.

In preferred embodiments, the oxidant is gaseous oxygen, which is transported to the biocathode via diffusion. In other preferred embodiments, the oxidant is a peroxide compound.

The biofuel cells of the embodiments can comprise (i) a bioanode comprising an immobilized organelle; and a biocathode comprising an immobilized organelle; (ii) a bioanode comprising an immobilized organelle and a biocathode comprising an immobilized enzyme; (iii) a bioanode comprising an immobilized enzyme and a biocathode comprising an immobilized organelle; and (iv) a bioanode comprising an immobilized organelle and a cathode; and (v) a biocathode comprising an immobilized organelle and an anode.

The biofuel cell of the instant invention may comprise a polymer electrolyte membrane (“PEM” or salt bridge, e.g., Nafion® 117) to separate the anode compartment from the cathode compartment. However, for embodiments having a bioanode and a biocathode, a PEM is not necessary and a membraneless biofuel cell is produced. The preferential selectivity of the organelles used in the bioanode and biocathode for catalysis of either the oxidant or the fuel fluid reaction makes the physical separation of the anode compartment from the cathode compartment unnecessary.

### II. Microfluidic Biofuel Cell

Among the various aspects of the invention is a microfluidic biofuel cell utilizing a fuel fluid to produce electricity via enzyme mediated redox reactions taking place at micromolded microelectrodes with immobilized organelles therein. As in a standard biofuel cell, the bioanode is the site for an oxidation reaction of a fuel fluid with a concurrent release of electrons. The electrons are directed from the bioanode through an electrical connector to some power consuming device. The electrons move through the device to another electrical connector, which transports the electrons to the biofuel cell's biocathode where the electrons are used to reduce an oxidant to produce water. In this manner, the biofuel cell of the present invention acts as an energy source (electricity) for an electrical load external thereto. To facilitate the fuel fluid's redox reactions, the microelectrodes comprise an electron conductor, an organelle, and an organelle immobilization material.

Unlike a standard biofuel cell, however, the biofuel cell of the invention utilizes at least one micromolded electrode. In one embodiment, the micromolded electrode has a flow through structure that allows fuel to flow within the microelectrode. When compared to conventional biofuel cell electrodes, this structure yields a higher current density because of the higher amount of microelectrode surface area in contact with the fuel. In another embodiment, the micromolded electrode has an irregular topography. Again, the current density of the microelectrode is greater than conventional biofuel cell electrodes because of a higher amount of surface area in contact with the fuel. These features combine with other features disclosed herein to create a biofuel cell with increased current density over conventional biofuel cells from a dimensionally smaller source. Finally, the method of the current invention can advantageously be used to economically produce disposable fuel cells.

A. Microfluidic Channel

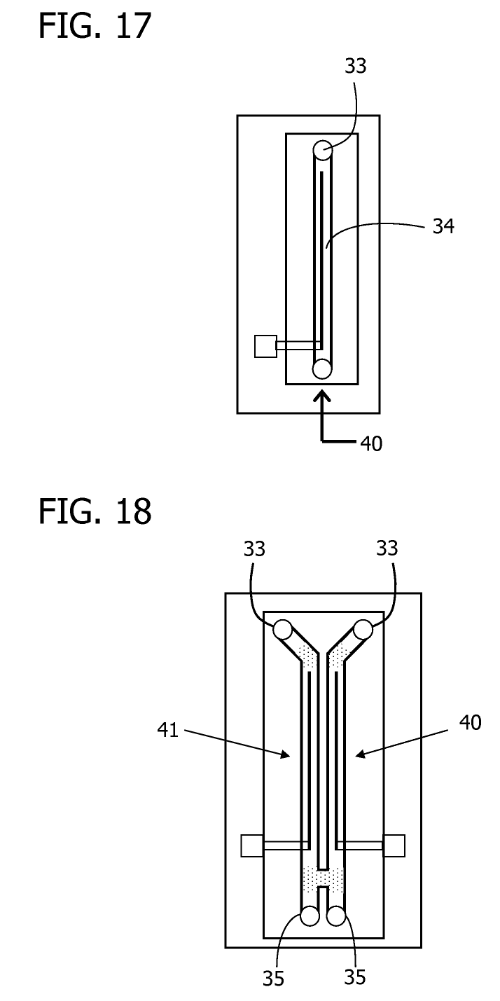

Beyond the bioanode and/or biocathode, the microfluidic biofuel cell is characterized by at least one microfluidic channel that, in service, houses the bioanode and/or the biocathode, the fuel fluid, and the oxidant. The microfluidic channel's configuration can vary depending on the application. In one embodiment, the microfluidic channel can simply be a rectangular chamber with the bioanode and/or the biocathode of the biofuel cell contained therein. See FIG. 17. In other embodiments, the configuration of the microfluidic channel can be more elaborate for any desired purpose, such as to ensure that the bioanode solution and the biocathode solution do not come into physical contact with one another. See FIG. 18.

With reference to FIGS. 17 and 18, the fuel fluid and/or oxidant flow through the microfluidic channel (34), over or through the microelectrode(s), from one end of the microfluidic channel (entry) (33) to the opposite end (exit) (35). In FIG. 18, the bioanode is represented by (41) and the biocathode is represented by (40). The microfluidic channel facilitates convective flow of the fuel fluid and/or oxidant over the microelectrode(s) while preventing leakage of the same outside the microfluidic channel (34).

B. Electrical Connectors

The electrical connectors provide electrical contact from the microelectrodes to the electrical load external to the microfluidic biofuel cell. In the most general sense, the electrical connector can be any material and structure that facilitates the transfer of electrons from the bioanode to the electrical load and back to the biocathode. In one preferred embodiment, the electrical connector of the microfluidic biofuel cell provides attachment leads to which another device can make physical and electrical contact. This other device, e.g. copper wire, then transports electrons to and from the external electrical load.

In one preferred embodiment, the electrical connector is a thin layer connector that is formed on the microfluidic biofuel cell's substrate prior to other processing. In this embodiment, the subsequently formed microelectrodes are arranged such that they intersect their respective electrical connectors. In an alternative embodiment, the electrical connector is a cylindrical body of electrically conductive material that is attached to the microelectrodes subsequent to their processing.

### III. Microfluidic Biofuel Cell Fabrication

In fabricating a microfluidic biofuel cell in accordance with this invention, a substrate is used on which the other biofuel cell components are constructed. In a preferred embodiment, the first step is to form the electrical connectors, followed by the fabrication of the microelectrodes, and the optional step of defining a biofuel chamber. In an alternative embodiment, the electrical connectors are formed subsequent to the other features.

A. Fabrication of Electrical Connectors

The microfluidic biofuel cell of the invention is formed by providing a substrate onto which the remaining components are formed. The substrate can be made of any material that is not conductive, will not passivate the conductive material of the microelectrode, to which the conductive material will adhere throughout processing, and to which molds can be reversibly sealed. In one embodiment, the substrate is glass. In a preferred embodiment, the substrate is poly(dimethylsiloxane) (PDMS). In another preferred embodiment, the substrate is polycarbonate. In one embodiment, the substrate is flat. In alternative embodiments, the substrate can take on a geometric shape that advantageously suits the particular application.

In a preferred embodiment, the first biofuel cell feature formed on the substrate is an electrical connector, which will be in electrical contact with the microelectrodes in the completed biofuel cell to provide the means for connecting the external electrical load to the microelectrodes. The connector can be made of any electrically conductive material. Exemplary materials include platinum, palladium, gold, alloys of those precious metals, carbon, nickel, copper and stainless steel. In one embodiment, the connector is made of platinum. In various preferred embodiments, the connector is made of carbon ink.

The connector can be formed on the substrate using conventional photolithographic techniques known in the silicon wafer industry. For example, to form a thin layer platinum electrical connector, a titanium adhesion layer is first sputtered onto the substrate. This is followed by sputtering a layer of platinum over the titanium layer. Both sputtering processes can be carried out, for example, in an argon-ion sputtering system. The connectors will then be defined by photolithography, with photoresist applied to the platinum layer to protect the desired connector locations. Chemical etching of the two layers with commercially available etchants followed by stripping of the photoresist will yield the finished platinum electrical connectors. In an alternative embodiment, the electrical connectors are the last feature formed. This embodiment is detailed below.

B. Fabrication of Microelectrodes

Following the creation of electrical connectors on the biofuel cell's substrate, the next step is the fabrication of the bioanode and the biocathode. These can be formed in succession or simultaneously.

1. Bioanode Fabrication

In one embodiment, the bioanode and the biocathode are formed on the substrate in succession, where the order of formation is not critical. For the purposes of presentation only, the bioanode fabrication will be detailed first. The first step of fabricating a microscale bioanode is creating a pattern of a microchannel in the surface of a casting mold. In general, the casting mold can be made of any material that is not conductive, will not passivate the conductive material and is able to be reversibly sealed to the substrate, with exemplary materials including silicon, glass, and polymers. The casting mold is preferably made of a polymer, even more preferably made of PDMS. Most preferably, the casting mold is made of polycarbonate.

In a preferred embodiment where the casting mold is a polymer, the pattern is created by using known soft lithography techniques to produce the microchannel in the casting mold to define the shape and size of the bioanode. Soft lithography techniques generally entail the process of molding a prepolymer against a lithographically-defined master that has a raised image of the desired design. The soft lithography technique employed should be able to yield microchannels in the casting mold between about 1 μm to about 1 mm, between about 1 μm to about 200 μm, preferably between about 10 μm to about 200 μm, more preferably between about 10 μm to about 100 μm, and most preferably as small as about 10 μm or less. Exemplary soft lithography techniques include near-field phase shift lithography, replica molding, microtransfer molding (μTM), solvent-assisted microcontact molding (SAMIM), and microcontact printing (μCP). Preferably, the microchannels are formed using replica molding.

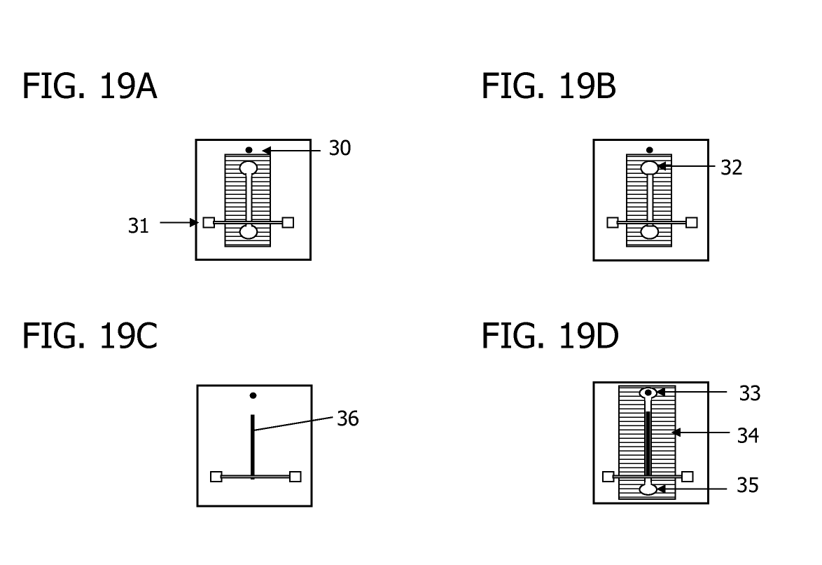

After the microchannel is formed in the casting mold, the patterned side of the casting mold is adhered to the substrate to complete the mold of the microelectrode. See FIG. 19(a). In the embodiment where the electrical connector (31) has previously been formed on the substrate, the microchannel should align over the electrical connector such that the finished microelectrode will be in electrical contact with the connector. Further, a tubing connector (30) is adhered to the substrate to maintain the position that will later become the entry reservoir.

Next, with reference to FIG. 19(b), an electron conductor solution is flowed into the casting mold's microchannel through an entry reservoir (32) that has been created in the casting mold at one end of the microchannel. This entry reservoir (32) is analogous to a pouring basin in the traditional art of metal casting. Excess solution will exit the microchannel at a vent located at the end of the microchannel opposite the entry reservoir.

The electron conductor solution can be any solution that comprises an electron conductor source and a liquid carrier that can be removed via curing to yield a solid microelectrode. The numerous potential electron conductor materials are listed above in I.A.1. In one preferred embodiment, the electron conductor source is a carbon source. In a more preferred embodiment, the electron conductor source is a carbon-based ink. In one such embodiment, the liquid carrier is a carbon-based ink thinner, e.g., Ercon N160 Solvent Thinner. Depending on the nature of the liquid carrier in the solution, two types of microelectrode structures can be formed according to the invention—solid microelectrodes or flow through microelectrodes. With lower viscosity liquid carriers, solid microelectrodes are produced. These microelectrodes are substantially continuous and solid, and fuel fluid flows over such microelectrodes during use. With higher viscosity liquid carriers, flow through microelectrodes are produced with a structure enabling fuel fluid to flow therethrough during use, effectively increasing the surface area of the microelectrode in contact with the fuel fluid.

Regardless of the particular structure, a microelectrode formed in accordance with this invention has several advantages over microelectrodes formed using traditional processes, which necessarily have flat topography. As such, any fluid flowing over conventional microelectrodes has a generally regular flow pattern and is in contact with a generally defined amount of microelectrode surface area. This flat geometric surface area is calculated by adding the rectangular surface area of the top and sides of the flat microelectrode. As current production of a microelectrode is determined in large part by the surface area in contact with the fuel fluid, a flat microelectrode's current production capabilities can only be increased by increasing its size. In contrast, microelectrodes formed in accordance with this invention have highly irregular, three dimensional topography, which yields at least two distinct advantages. First, the effective surface area of the invention's microelectrode is substantially increased compared to a flat screen printed microelectrode. The effective surface area of the microelectrodes herein described is the sum of surface area of the individual peaks and valleys characterizing the microelectrode's topography. One accurate method of calculating this effective surface area is to compare the current output of a microelectrode formed according to the invention with a flat microelectrode of the same length, width, and height dimensions. For example, such analysis of microelectrodes has shown current output of 9.85×10⁻⁴ A/cm² for a microelectrode of this invention, compared to 2.06×10⁻⁴ A/cm² for a conventional glassy carbon electrode. Further, the microelectrode's irregular topography can create turbulent flow of the fluid. Such a flow pattern is advantageous because it induces mixing of the fluid over the microelectrode, which in turn increases the transport rate of the fluid to the microelectrode. Increasing the transport rate of the fluid facilitates the reactions taking place within the microelectrode, thereby increasing the microelectrode's current load capability.

In one alternative embodiment, a primer is flowed into the casting mold's microchannels and quickly dried prior to introducing the electron conductor solution. The primer can be any material that will help prevent the electron conductor from becoming semi-permanently attached to the casting mold. For example, in the carbon-based ink embodiment, carbon-based ink thinner can be used as a primer, if one is desired.

After the solution fills the casting mold's microchannels, heat is applied to cure the electron conductor solution. In general, heating should be conducted at a temperature sufficient to remove the liquid carrier from the solution, but low enough so that the resulting microelectrode is not damaged. In one preferred embodiment, heating occurs at about 75° C. Also, heat should be applied for a time sufficient to remove substantially all of the liquid carrier from the solution. In one preferred embodiment, heat is applied for at least about one hour. In another preferred embodiment, heating occurs at about 75° C. for about one hour. With reference to FIG. 19(c), the curing process yields a solidified microelectrode (36) that is approximately 20% smaller than the original size of the casting mold's microchannel(s) due to evaporation of the carrier.

In the method according to the invention, the microelectrode is treated to impart an organelle, and an organelle immobilization material thereto to form a bioanode. In certain embodiments, the organelle immobilization material containing the organelle is applied to the cured microelectrode. To form the bioanode, the casting mold is removed from the substrate after curing the microelectrode. See FIG. 19(c). With reference to FIG. 19(d), in place of the casting mold, a gas-permeable mold with a microchannel (34) approximately twice the width of the casting mold's microchannel is reversibly sealed over the microelectrode. The gas-permeable mold can be made of any material that is not conductive, will not passivate the electron conductor and facilitates evaporation of a solvent. Preferably, a silicon polymer, such as PDMS, is used as the gas-permeable mold material. More preferably, a thermoplastic resin, such as polycarbonate, is the gas-permeable mold material. After the gas-permeable mold is in place, an organelle immobilization material containing a bioanode organelle is applied to the cured microelectrode. This is accomplished by syringe pumping the casting solution into the entry reservoir (33) and through the gas-permeable mold to an exit vent (35). See FIG. 19(d) for a finished bioanode.

In all embodiments, the specific composition of the organelle immobilization material and the organelle is detailed above in I.A.4.-I.A.5. Preferred organelle immobilization materials for the bioanode is a tetraalkyl ammonium-modified perfluoro sulfonic acid-PTFE copolymer or a hydrophobically modified polysaccharide, particularly, a tetraalkyl ammonium-modified perfluoro sulfonic acid-PTFE copolymer. The preferred organelle at the anode is mitochondria. Also, the casting mold can include more than one microchannel in all embodiments.

2. Biocathode Fabrication

To form a biocathode in accordance with the invention, the same general processing steps taken to fabricate the bioanode can be used to produce a biocathode. The embodiments for treating the biocathode with the organelle immobilization material, and the organelle are the same as those for the bioanode. The specific composition of the organelle immobilization material, and the organelle is detailed above in I.B.4.-I.B.5. The preferred organelle immobilization material for the biocathode is a tetraalkyl ammonium-modified perfluoro sulfonic acid-PTFE copolymer or a hydrophobically modified polysaccharide. Additionally for the biocathode, the preferred organelle is chloroplast.

3. Forming the Operational Biofuel Cell

After the bioanode and biocathode have been formed in accordance with this invention, the casting or gas-permeable molds are optionally removed. In this optional embodiment the bioanode and biocathode remain on the substrate. After the casting or gas-permeable molds are removed, a microfluidic channel form is aligned over the bioanode and biocathode. This form is micropatterned so as to create at least one microfluidic channel through which the biofuel cell's fuel fluid can flow. The form can be made of any material that is not conductive, will not passivate the conductive material and will adhere to the substrate. Preferably, the form is PDMS. More preferably, this overlay is polycarbonate. The micropatterns of the microfluidic channel(s) in the form can be created by using any known soft lithography technique. In one embodiment, the microfluidic channel is about two to four times larger than the microelectrodes. In another embodiment, the microfluidic channel is approximately the same size as the microelectrodes. The microfluidic channels of the form essentially define the electrochemical cell in which the fuel fluid will interface with the microelectrodes. When only one microfluidic channel is used to house the bioanode, biocathode, fuel fluid, and oxidant, the mixture of fuel fluid and oxidant in the same microfluidic chamber does not compromise the function of the microelectrodes of the invention because their redox reactions are selective. Stated another way, the bioanode will only react with fuel fluid and the biocathode will only react with the oxidant, and no cross reaction takes place.

In an alternative embodiment, the casting or gas-permeable mold(s) remain in contact with the substrate and serves to define the microfluidic channels of the biofuel cell, acting as the microfluidic channel form described above. In this embodiment, the fuel fluid travels through the space between the microchannels of the mold(s) and the bioanode or biocathode. In this embodiment, subsequent processing must be performed to create a junction between the individual bioanode and biocathode microfluidic channels. To form the junction, a passage connecting the individual microfluidic chambers is formed in the mold(s) by any appropriate means, such as applying a perpendicular force to the top of the mold(s) or removing sufficient material from the mold(s). Thereafter, the passage is covered by a material that will seal the junction to inhibit leakage of the fuel fluid or oxidant during operation. The material must be capable of being joined to the mold material to create the appropriate seal. In one embodiment, the covering material is simply a flat piece of the mold material, such as PDMS or polycarbonate.

4. Optional Formation Embodiments

The microelectrode fabrication technique described above in III.B.1. refers to the embodiment wherein the bioanode and the biocathode were formed successively, which was followed by a method of connecting the bioanode and biocathode via microchannels to form the biofuel cell. In an alternative embodiment, the bioanode and the biocathode can be formed simultaneously. In this embodiment, a single casting mold is patterned to form both the bioanode and biocathode. Alternatively, a combination of casting molds can be used to form the individual bioanode and biocathode. In either case, after the bioanode and biocathode are simultaneously formed, the operational biofuel cell is formed by either applying a microfluidic channel form or modifying the casting mold(s) as detailed above in III.B.3.

The embodiment described above in III.A. describes the formation of the electrical connectors on the substrate prior to other processing steps. In an alternative embodiment, the electrical connectors are added to the microfluidic biofuel cell as a final processing step. Here, holes are created in the microfluidic channel form or the modified casting mold(s) to expose a portion of each bioanode and biocathode. Next, electrical connectors are physically joined to the exposed portion of each bioanode and biocathode. In this embodiment, the electrical connectors can be any material in any structure that will enable the external electrical load to make electrical contact with the bioanode and biocathode. In one preferred embodiment, the electrical connectors are cylindrical copper bodies. Further, any joining technique capable of maintaining the electrical contact between the electrical connectors and the bioanode and biocathode can be employed. In one preferred embodiment, silver epoxy paste can be used to join the electrical connectors and the bioanode and biocathode electrically. This embodiment has the advantage of increasing the conductivity between these components.

The above embodiments have described a biofuel cell wherein both the bioanode and the biocathode are housed within the microchannel(s) of the biofuel cell. While this is the preferred embodiment, alternative embodiments of the invention include an anode or a cathode located external to the microchannel(s) of the biofuel cell. Here, a fuel cell is formed by combining a microfluidic bioanode or biocathode with the appropriate external anode or cathode.

C. Use of the Microfluidic Biofuel Cell

After fabrication of the operational microfluidic biofuel cell of this invention is complete, it can be utilized in myriad applications where a fluid fuel source and oxidant are available for the bioanode and biocathode respectively. In use, the fuel fluid and the oxidant travel through the microfluidic channel(s) to contact the bioanode and biocathode. There, the redox reactions described above at I. take place to create a current source. The microfluidic biofuel cell of the instant invention may be used in any application that requires an electrical supply, such as electronic devices, commercial toys, internal medical devices, and electrically powered vehicles. Further, the microfluidic biofuel cell of the instant invention may be implanted into a living organism, wherein the fuel fluid is derived from the organism and current is used to power a device implanted in the living organism.

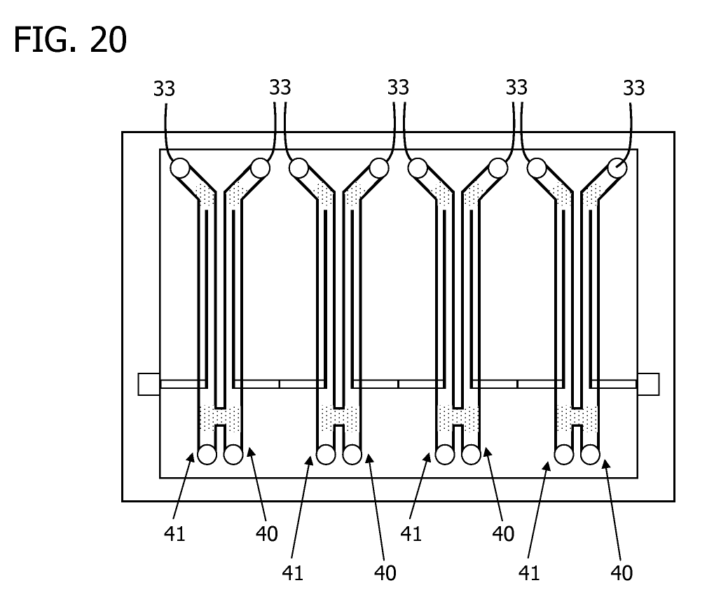

In addition, multiple microfluidic biofuel cells of the invention can be joined in a series electrical circuit to form a biofuel cell stack. See FIG. 20. A series stack is formed by electrically joining the bioanode (41) of one biofuel cell to the biocathode (40) of another biofuel cell, which is in turn connected to another bioanode (41) until the desired stack is obtained. Fuel fluid and/or oxidant flows into the microfluidic chamber in an entry reservoir (33). By forming stacks, the total voltage output of a microfluidic biofuel cell circuit is theoretically the sum of the voltage output from the individual microfluidic biofuel cells in series. The greater overall voltage output of such a stack is useful in supplying electricity to electronic devices, toys, medical devices, and vehicles with power requirements higher than an individual microfluidic biofuel cell could provide.

### IV. Methods of Generating Electricity

The invention includes a method of generating electricity comprising oxidizing the fuel fluid at the anode and reducing the oxidant at the cathode, wherein the electricity is generated using a biofuel cell comprising the bioanodes and/or biocathodes as described above.

V. Uses of Electrodes with Immobilized Mitochondria

As wireless internet networks have become more prevalent over the last decade, there has been a push for wireless sensor networks (WSNs) as well. Wireless sensor networks involve a series of small autonomous sensors spread throughout an area that can monitor conditions and wirelessly transmit this information back to a central location. Each wireless sensor package typically contains a sensor, a battery, a microcontroller, and a radio communication device to transmit the data. These type of networks have great applicability for defense, energy, and homeland security applications, but currently the deployment of these sensors is challenged by a couple of key limitations. One limitation is the specificity and sensitivity of the sensor used, in this case a sensor or biosensor for explosives. Improved specificity and sensitivity is needed to ensure reliable responses (e.g., decrease false positive signals and increase detection limits). Another limitation is the power source (batteries) used to control both the sensor and the radio communication device. Currently, there is a power drain on the operation of the wireless sensor even when it is not transmitting a signal to the central location due to the constant power needed to operate the sensing element. This constant power drain limits the lifetime of the battery. Therefore, in order for wireless sensor networks to be feasible for defense, energy and homeland security applications, power sources must be developed that can be replaced or recharged less frequently.

One approach to long-term explosives sensing for wireless sensor networks is self-powered sensors. Conventional self-powered sensors combine traditional sensor and battery components together; they do not produce electrical power in the absence of an analyte, but produce sufficient electrical power after exposure to the analyte to power an advisory signal such as an alarm. Another aspect of the present invention is to use a mitochondria-based biofuel cell of the invention as a self-powered sensor for nitroaromatic explosives.

As with any fuel cell, a mitochondrial fuel cell will only produce electrical energy in the presence of fuel, but mitochondria are different than most traditional catalysts in that there are a number of agents (e.g. the antibiotic oligomycin) that can inhibit mitochondria from functioning, which in turn will delay or minimize electrical power generation. However, this mitochondrial function (metabolism of fuel) can be returned by the addition of an uncoupler or decoupler. It is important to note that nitroaromatic compounds are common explosive materials, but are also selective decouplers for mitochondrial inhibition. Therefore, a biofuel cell of the present invention as described herein, when used as a self-powered sensor for nitroaromatic explosives, includes a bioanode comprising mitochondria as the organelle and an agent which inhibits the enzyme from reacting with the fuel fluid until the mitochondria is exposed to the nitroaromatic explosive. The biofuel cell is a self-powered sensor because no power or not enough power is produced in the absence of the explosive material to produce an alarm signal, but after the nitroaromatic explosive is present, the inhibited mitochondria is decoupled and will catalyze the oxidation of fuel, such as pyruvate, to carbon dioxide. This oxidation at the anode of a biofuel cell in combination with the reduction of oxygen to water at the cathode will produce power that can then be used for signaling the presence of the explosive.

Various inhibiting agents can be used such as oligomycin, valinomycin, antimycin, ossamycin, cytovaricin, and combinations thereof

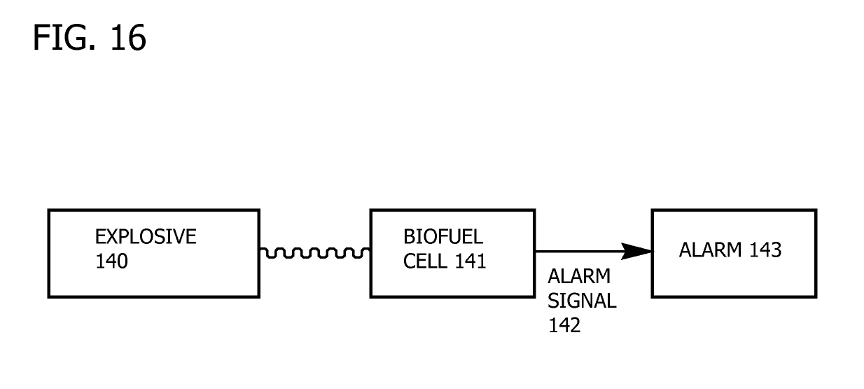

These biofuel cells can be tailored to produce appropriate power for signaling (e.g., radio signal, or a visual or audible alarm) by altering electrode size, ordering biofuel cells in series or parallel configurations, and altering the fuel concentration. As depicted in FIG. 16, a self-powered explosives sensor would involve contact of a biofuel cell 141 with an explosive agent 140. The biofuel cell 141 would in turn produce an alarm signal 142 for use with conventional circuitry for producing the alarm 143. The alarm 143 can be a speaker, buzzer, optical indicator such as an LED, or combinations thereof. In an alternate embodiment, the alarm signal 143 output powers a radio transmitter that produces an audible alarm.

A light sensor is a sensor that measures the amount of light that it sees. It reports the amount of light by sending a light signal to a detector. Thus, a light sensor can determine whether it is seeing a white piece of paper or a black piece of paper by sending a different light signal to the detector. Light sensors having thylakoids immobilized on an electrode could be used to detect light variations in many different settings. Also, a light sensor electrode could be powered by pairing it with an anode to form a biofuel cell with the immobilized thylakoid at the cathode.

Immobilized chloroplasts could also be used to produce carbohydrates. In an organism, chloroplasts absorb sunlight and use it along with water and carbon dioxide to produce carbohydrates. Similarly, immobilized chloroplasts could produce carbohydrates from water and carbon dioxide by absorbing light energy.

Immobilized mitochondria or mitoplasts could also be used to regenerate ATP from ADP and NADH from NAD⁺. In organisms, regenerating these species is a function of mitochondria and the immobilized mitochondria or mitoplasts could act in a similar manner.

Further, various mitochondrial diseases result from failures of the mitochondria. Mitochondria are responsible for creating more than 90% of the energy needed by the body to sustain life and support growth. When they fail, less and less energy is generated with the cell. Cell injury and even cell death can follow. If this process is repeated throughout the body, whole systems can fail, and the life of the patient is severely compromised. By administering a therapeutic immobilized mitochondria to a patient in need thereof, an improvement in mitochondrial function may result because the mitochondria is protected from various agents by the immobilization material.

### Definitions

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The terms “halogen” or “halo” as used herein alone or as part of another group refer to chlorine, bromine, fluorine, and iodine.

The term “acyl,” as used herein alone or as part of another group, denotes the moiety formed by removal of the hydroxyl group from the group —COOH of an organic carboxylic acid, e.g., RC(O)—, wherein R is R₁, R₁O—, R₁R₂N—, or R₁S—, R₁ is hydrocarbyl, heterosubstituted hydrocarbyl, or heterocyclo, and R₂ is hydrogen, hydrocarbyl or substituted hydrocarbyl.

The term “acyloxy,” as used herein alone or as part of another group, denotes an acyl group as described above bonded through an oxygen linkage (—O—), e.g., RC(O)O— wherein R is as defined in connection with the term “acyl.”

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The following examples illustrate the invention.

### Examples

### Example 1

### Extraction of Mitochondria from Russet Potatoes

A homogenization buffer consisting of 100 mM Tris HCl (pH 8), 2.6 M NaCl, 50 mM ethylenediaminetetraacetic acid, 0.4% bovine serum albumin, 0.1% cystine, and 28 mM dithiothetol was prepared. The solution was mixed with the exception of the cystine and the dithiothetol, then chilled in the fridge. The cystine and the dithiothetol were mixed in right before use. Russet potatoes were quartered and immediately juiced in a kitchen juice extractor. The juice (100 mL) was collected in an equal amount of chilled homogenization buffer (100 mL). For the rest of the experiment the mitochondrial solution was kept at 4 degrees Celsius.

The juice and buffer were centrifuged at varying speeds and times wherein the supernatant was retained for the next centrifugation operation and the precipitate was discarded. The successive centrifuge speeds and times were (1) 500 rpm for 10 minutes; (2) 2600 rpm for 10 minutes; (3) 2600 rpm for 15 minutes; and (4) 15,557 rpm for 15 minutes. The final precipitate contained the mitochondria.

### Example 2

### Preparation of Bioanode Comprising Mitochondria

The wet mitochondria precipitate prepared in Example 1 was used directly. Wet precipitate (18.7 mg) was suspended in 1 mL of pH 7.15 phosphate buffer with 1 M NaCl. 100 uL of mitochondrial suspension was mixed with 100 uL tetrabutyl ammonium bromide modified Nafion® suspension. The resulting solution was pipetted onto 1 cm² carbon paper electrodes (66.6 uL each). The electrodes were placed into a vacuum chamber and kept there for approximately 10-20 minutes until dry.

### Example 3

### Testing of Bioanodes Comprising Mitochondria

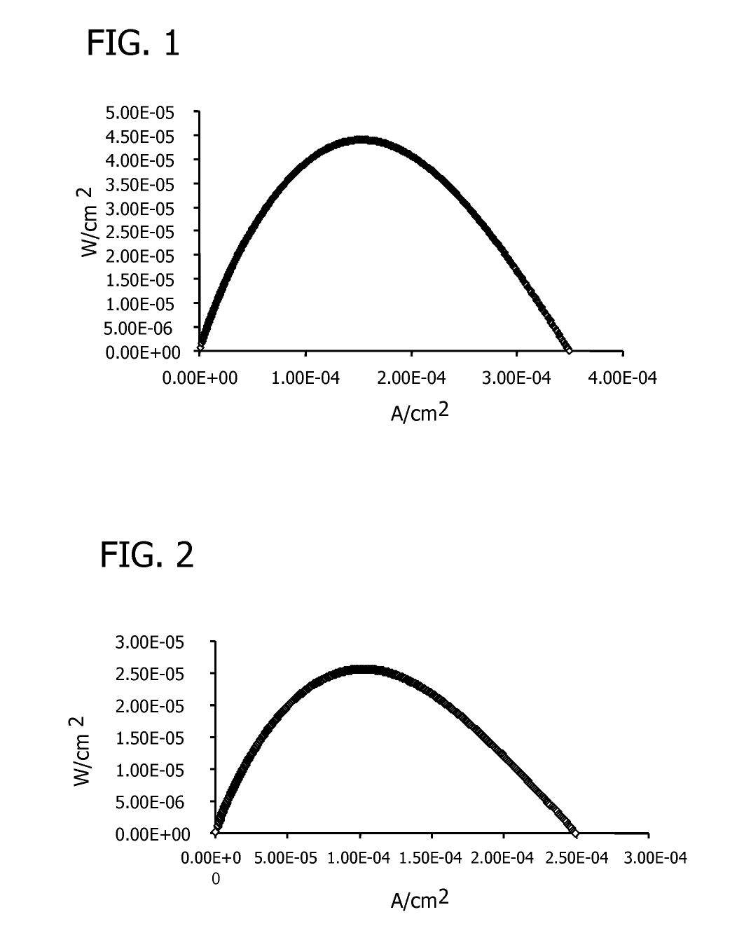

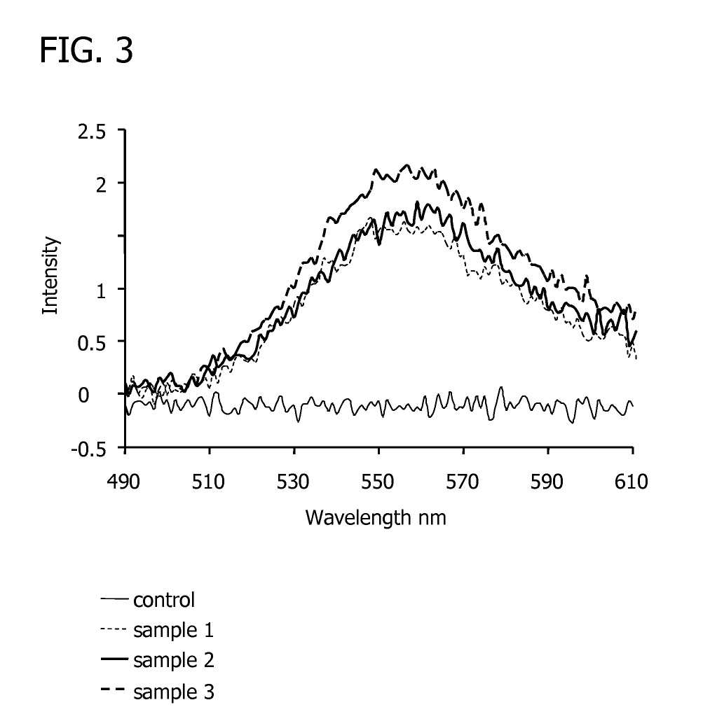

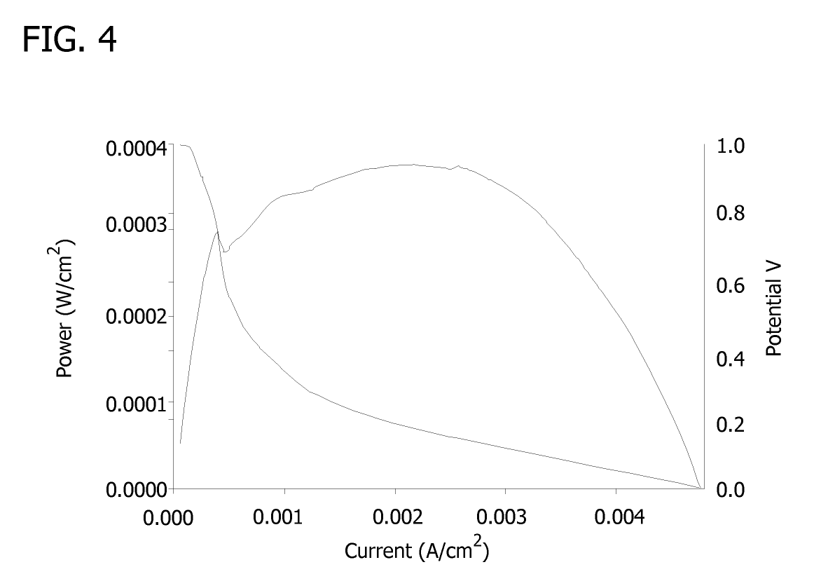

Test 1: The electrodes where tested in a standard H-cell with a platinum cathode and 100 mM glucose in pH 7.15 buffer with 1M NaCl. The bioanodes produced open circuit potentials between 0.607V and 0.672V and the maximum current from maximum load was between 0.201 mA cm² and 0.250 mA cm². See FIG. 1.

Test 2: The electrodes where tested in a standard H-cell with a platinum cathode and 100 mM pyruvate in pH 7.15 buffer with 1M NaCl. The bioanodes produced open circuit potentials between 0.632 V and 0.717 V and the maximum current from maximum load was between 0.250 mA/cm² and 0.349 mA/cm². See FIG. 2.

Test 3: An assay was done by casting 100 uL of the 50/50 modified Nafion-mitochondria solution in the bottom of a cuvette and allowing to dry in a vacuum chamber. The cuvette was then filled with 2 mL of 1 mM adenosine diphosphate and 1 mM pyruvate in pH 7.15 buffer and allowed to sit for 3 hours. Then 0.5 mL of a 0.1 mg/10 mL luciferin/luciferase pH 7.15 solution was added to the cuvette and tested for luminescence. The control with no cast membrane gave a reading of 0.0 for luminescence. The cuvettes with the cast membrane all gave readings of 2.0.

After assays showed that the mitochondria were viable after immobilization, carbon-13 NMR was also performed on the immobilized mitochondria to determine if complete oxidation of pyruvate (the natural substrate for mitochondria) was occurring in the immobilized mitochondria. The results indicated that only a pyruvate peak was detectable for the control experiment that did not contain immobilized mitochondria, but carbonate, bicarbonate, and carbonic acid peaks were present in the sample containing immobilized mitochondria. This showed that the immobilized mitochondria are capable of undergoing complete oxidation of carbon-13 labeled pyruvate to carbon dioxide. The pyruvate control solution which contained no mitochondria showed a carbon-13 NMR peak at 199 ppm, corresponding to pyruvate. The NMR spectra of the labeled pyruvate solution equilibrated with the immobilized mitochondria showed the pyruvate had been metabolized to carbon dioxide, as shown by peaks at 165 ppm, 163 ppm, and 152 ppm, corresponding to the carbonate, bicarbonate, and carbonic acid species, respectively.

### Example 4

### Mitochondrial Bioanode

After demonstrating that the immobilized mitochondria were still viable and capable of completely oxidizing fuel, the immobilized mitochondria were then used to fabricate a mitochondria-catalyzed pyruvate biofuel cell. The mitochondria were immobilized on a poly(methylene green) coated carbon paper electrode, which was employed as the anode of a pyruvate/oxygen biofuel cell. Within the mitochondria are all of the enzymes of the Kreb's cycle. The Kreb's cycle contains eight enzymes, but only four of those enzymes are oxidoreductases (enzymes responsible for catalyzing oxidation and reduction reactions). All four of the oxidoreductase enzymes are dehydrogenase enzymes that oxidize a reactant, but all four dehydrogenase enzymes are also dependent on a coenzyme that is reduced while the reactant is oxidized. Poly(methylene green) is a well-known electrocatalyst for NADH, NADPH, and FADH₂, which are the coenzymes produced in the Kreb's cycle as pyruvate is oxidized. Therefore, the pyruvate bioanode functions by pyruvate diffusing through the hydrophobically modified Nafion® membrane to the mitochondria, where the enzymes of the mitochondria will oxidize the pyruvate to carbon dioxide, while reducing the coenzymes NAD, NADP, and FAD to NADH, NADPH, and FADH₂. The reduced coenzymes will then diffuse to the poly(methylene green) modified carbon paper surface and be oxidized to regenerate the original coenzymes NAD, NADP, and FAD. A representative power curve and polarization curve of the mitochondria-based biofuel cell with a commercial platinum cathode is shown in FIG. 4. The average maximum open circuit potential obtained was 0.71V with an average maximum power density of 0.75±0.41 mW/cm². This can be compared to NAD-dependent enzymatic biofuel cells which typically produce 0.6 to 0.82V and power densities of 1.16 mW/cm² under the same conditions. Therefore, this new type of biofuel cell is producing power densities that are comparable to enzymatic biofuel cells, while allowing for complete oxidation of fuel like a microbial biofuel cell, which leads to significantly higher energy densities. This biofuel cell in the laboratory has shown a lifetime of 75 days with no statistical degradation in power and with tests on-going.

### Example 5

### Direct Electron Transfer (DET) of Immobilized Mitochondria

Mitochondria where immobilized within a modified Nafion® membrane that was directly placed onto a carbon electrode without a mediator to determine if the mitochondria could exhibit Direct Electron Transfer (DET) to the carbon electrode. These low surface area mitochondria modified electrodes generated average open circuit potentials of 1.048 Volts, indicating that direct electron transfer was occurring with good efficiency. The mitochondria where also incorporated into a high surface catalyst support matrix of multiwalled carbon nanotubes (MWCNT) and immobilized within modified Nafion®. These high surface area mitochondria modified electrodes displayed enhanced power densities compared to the low surface area mitochondria modified electrodes and higher average open circuit potentials of 1.151 Volts indicating that the multiwalled carbon nanotube support matrix increased the efficiency of the DET from the mitochondria by allowing more surface area to make contact to them.

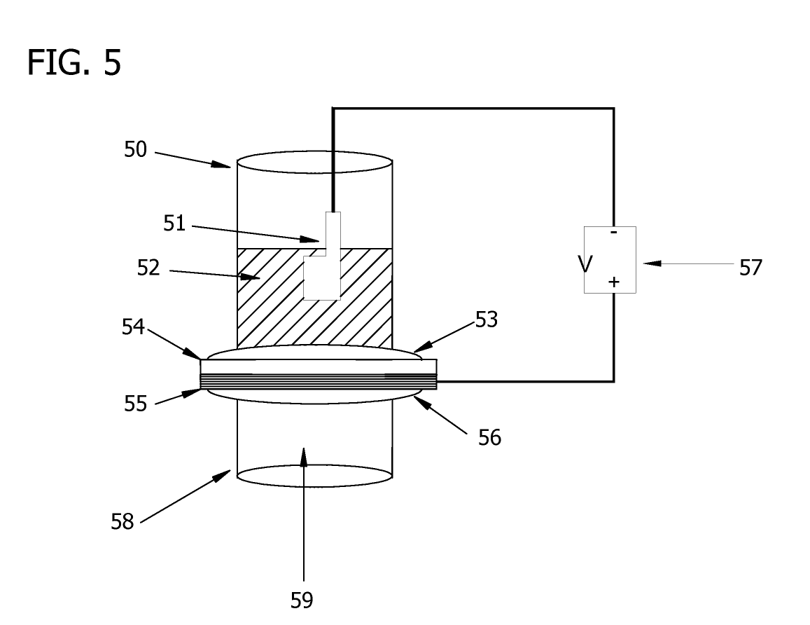

Low Surface Area Electrodes. Mitochondria were extracted from potatoes as described in Example 1. The mitochondrial pellet that was the product of the extraction was used directly. 18 mg of the wet mitochondria was added to a small plastic vial. In addition, 1 mg of adenosine diphoshphate (ADP) was added as well. To this vial 1 mL of pH 7.15 phosphate buffer was added, and the contents were thoroughly mixed. This mitochondria suspension was kept chilled until used. Toray paper purchased from E-TEK, model TGPH-060 was used as the carbon electrode backing. The Toray was cut into 1 cm² electrodes and placed into a weighboat. A mitochondria casting solution was prepared by taking 100 μL of mitochondria suspension and placing it into a small plastic vial. To this vial 100 μL tetrabutylammonium modified Nafion® (TBAB) suspension was added and the vial was mixed thoroughly. Then 50 μl of the mitochondria casting solution was cast onto each 1 cm² electrode. The electrodes where then dried in a low humidity environment overnight. Once dry the electrodes were soaked in pH 7.15 phosphate buffer that contained 6 M NaNO₃ electrolyte and 100 mM pyruvate for 48 hours to fully saturate them. The saturated electrodes were then tested in an I-Cell (see FIG. 5) with pH 7.15 phosphate buffer that contained 6 M NaNO₃ electrolyte and 100 mM pyruvate. An I-cell setup (FIG. 5) was used so that the fuel cell would be anode dependent and the platinum cathode would not be poisoned from being submerged in buffer solution. An I-cell allows the platinum electrode to operate in air breathing mode. FIG. 5 is a schematic of the I-cell that was used in this experiment. In FIG. 5, a glass tube 50 contains the fuel solution 52 and the bioanode 51 that is immersed in the fuel solution. The glass tube 50 is connected by O-ring 53 to a Nafion® polyelectrolyte membrane 54 and the fuel solution 52 also contacts the Nafion® polyelectrolyte membrane 54. The Nafion® polyelectrolyte membrane 54 is in contact with a 20% platinum gas diffusion electrode cathode 55 that is connected to another glass tube 58 using and O-ring 56. Air 59 can contact the 20% platinum gas diffusion electrode cathode 55 and there is an electrical connection from the cathode 55 to the bioanode 51 through a potentiostat 57.

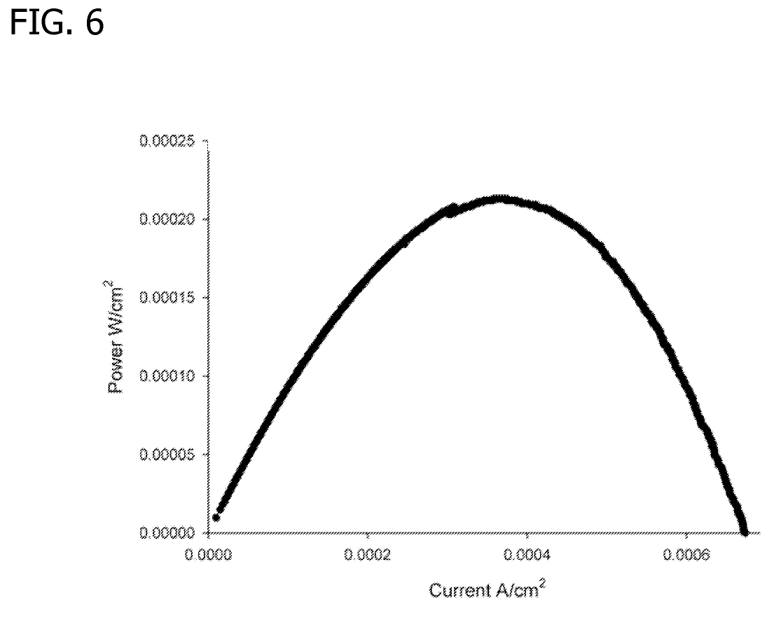

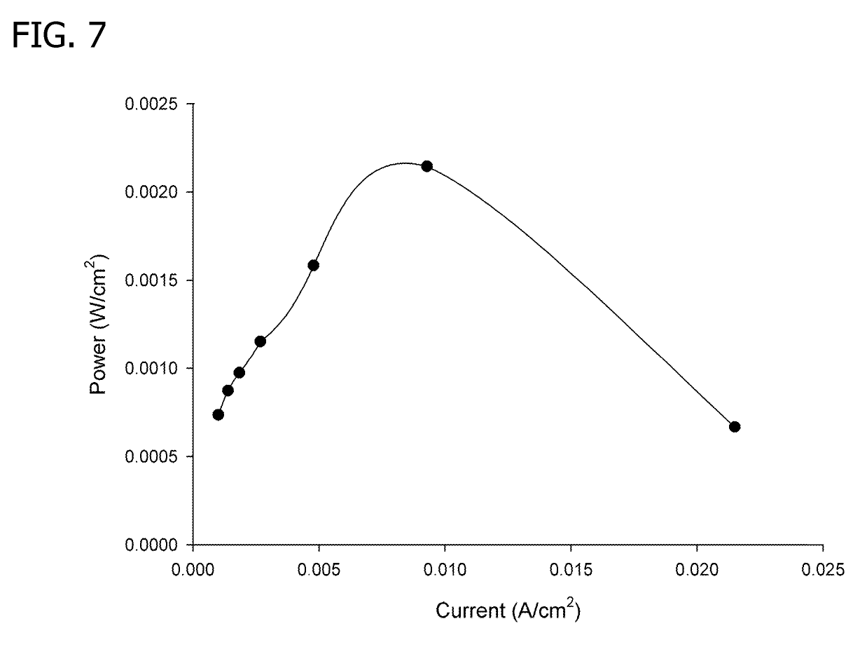

The low surface area mitochondria modified electrodes showed average open circuit potentials of 1.048±0.026 Volts. The average maximum diffusion-limited current was 0.222±0.089 mA/cm². The average maximum steady state power density was 0.0229±0.0056 mW/cm². The average maximum peak current was 18.10±3.63 mW/cm². The average maximum peak power density was 1.815±0.343 mW/cm². Representative power curves are shown in FIGS. 6 and 7.

High surface area electrodes. Mitochondria were extracted from potatoes as described in Example 1. The mitochondrial pellet that was the product of the extraction was used directly. 18 mg of the wet mitochondria was added to a small plastic vial. In addition, 1 mg of adenosine diphoshphate (ADP) was added as well. To this vial 1 mL of pH 7.15 phosphate buffer was added, and the contents were thoroughly mixed. This mitochondria suspension was kept chilled until used. Toray paper purchased from E-TEK, model TGPH-060 was used as the carbon electrode backing. The Toray was cut into 1 cm² electrodes and placed into a weighboat. Carboxylic acid functionalized MWCNTs purchased from CHEAPTUBESINC were used as the high surface area catalyst support matrix. MWCNTs (5 mg) was placed into a small glass vial. To this vial 200 μL of deionized water was added along with several ceramic beads to aid in dispersion of the MWCNTs. The contents of this vial were mixed thoroughly on a vortex mixer. Once fully dispersed, 100 μL of the mitochondria suspension was added directly to the dispersed MWCNTs. The contents were mixed well and then 100 μL of the mixture was applied directly to each 1 cm² Toray electrode. The electrodes were allowed to dry until the MWCNT layer was damp in a vacuum desiccator for 10-15 minutes. The electrodes were removed and 50 μL of TBAB was cast onto each 1 cm² electrode and allowed to soak into the support matrix. The electrodes were then allowed to dry fully in a low humidity environment overnight. Once fully dry, the electrodes were allowed to soak for 48 hours in pH 7.15 phosphate buffer that contained 6 M NaNO₃ and 100 mM pyruvate to ensure they were fully saturated. Once fully saturated, the electrode were tested in an I-Cell with pH 7.15 phosphate buffer that contained 6 M NaNO₃ electrolyte and 100 mM pyruvate.

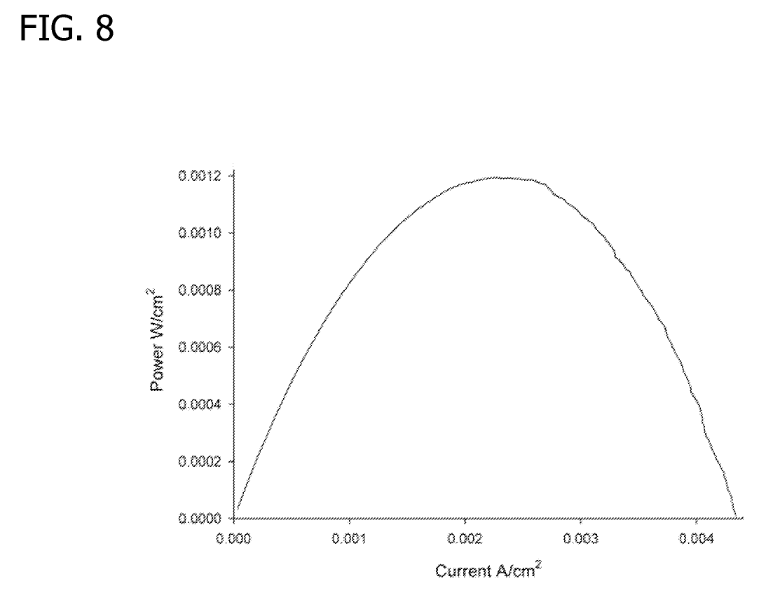

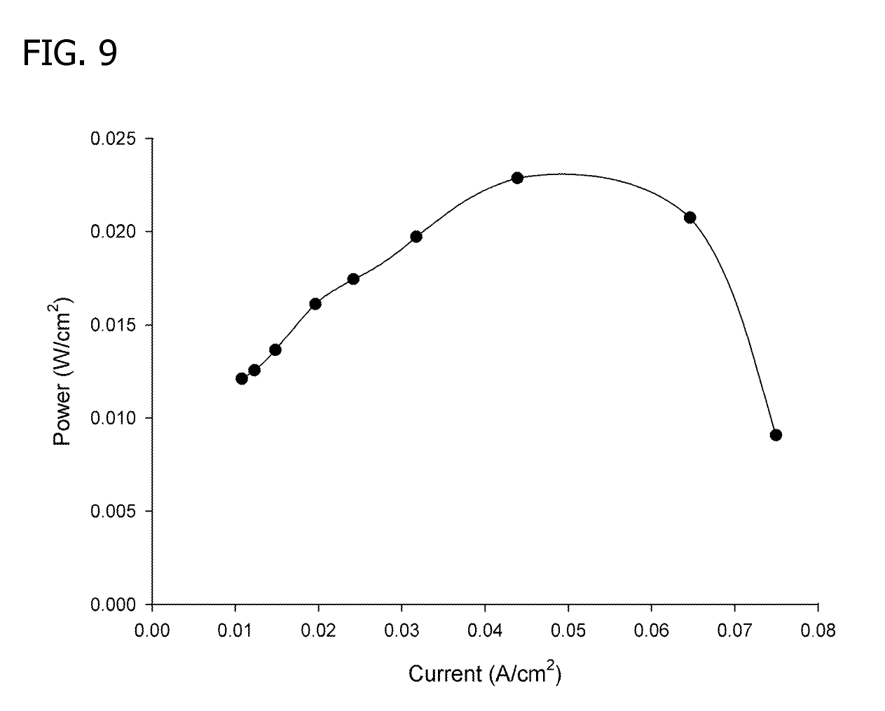

The high surface area mitochondria modified electrodes showed average open circuit potentials of 1.151±0.049 Volts. The average maximum diffusion limited current was 4.410±0.312 mA/cm². The average maximum steady state power was 0.745±0.412 mW/cm². The average maximum peak current was 72.92±2.35 mA/cm². The average maximum peak power density was 20.93±2.59 mW/cm². Representative power curves are shown in FIGS. 8 and 9.

Mitochondria were extracted from potatoes as described in Example 1. The mitochondria stock suspension was made by taking 22 mg of the wet mitochondrial pellet and suspending it in 1 mL of pH 7.15 phosphate buffer in a small glass vial. ADP (1 mg) was added to the vial and the mixture was mixed thoroughly. In a separate vial, 5 mg of carboxylic acid functionalized multiwalled carbon nanotubes (MWCNTs) was added followed by 200 μL of deionized water. This vial containing the MWCNTs was mixed vigorously until the MWCNTs were thoroughly dispersed. Then, 100 μL of mitochondria stock solution was added to it and it was mixed vigorously once again for 30 seconds. Then, 5 μL of this mixture was placed onto glassy carbon electrodes (GCE) and allowed to dry in a low humidity environment. Once dry 2 μL of TBAB modified Nafion® was placed on top of the MWCNT layer and allowed to dry in a low humidity environment. Then, six of the glassy carbon electrodes were soaked overnight in pH 7.15 phosphate buffer and another six were allowed to soak in pH 7.15 phosphate buffer with 100 mM pyruvate. Cyclic voltammograms (CV) were then run on each (GCE) in their respective soaking solutions. The CV's scan window was 0.5 Volts to −1.0 Volts with a scan rate of 0.1 V/s, and used a calomel reference electrode and a platinum mesh counter electrode.

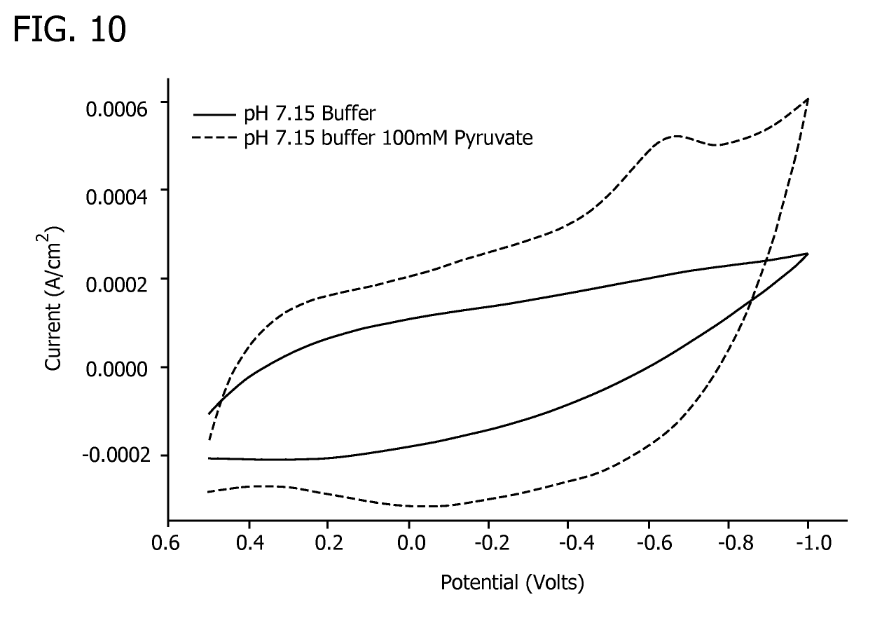

The CVs of the mitochondria showed significantly more current when pyruvate was present than when it was not as shown in FIG. 10. This indicated that metabolism of pyruvate was occurring and also that DET was occurring when pyruvate was present. Since there was no mediator present in the samples and it is known that NADH will not undergo direct electron transfer, this data supports that the mitochondria are intact and achieving direct electron transfer to the carbon electrode.

### Example 6

### Mitochondria-Based Nitroaromatic (Explosives) Self-Powered Sensor

Mitochondria were extracted from potatoes via the procedure described in Example 1. The wet mitochondrial pellet was used directly to make a stock suspension in pH 7.15 phosphate buffer that contained 18 mg/mL mitochondria, 1 mg/mL ADP, and 100 mM NaNO₃. This mitochondria suspension (100 μL) was mixed with 100 μL of TBAB modified Nafion® in a vial on a vortex mixer for 30 seconds. The resulting mitochondria/TBAB modified Nafion® suspension was then cast on 1 cm² Toray paper electrodes (50 μL each). The electrodes were then allowed to dry in a low humidity environment overnight. Once dry, the electrodes were saturated for 48 hours in a pH 7.15 phosphate buffer that contained 1 mg/mL ADP, 100 mM pyruvate, and 6 M NaNO₃. After saturation, the electrodes were tested in a pH 7.15 phosphate buffer solution containing 6 M NaNO₃ and 100 mM pyruvate on a model 650a CH Instruments potentiostat to determine their uninhibited performance in an I-Cell. The electrodes were then soaked for 12 hours in a 0.2 mg/mL oligomycin A/B/C-60/30/10 solution that was made by taking 1 mg of oligomycin A/B/C and dissolving it in 100 μL ethanol and adding it to 5 mL pH 7.15 phosphate buffer that contained 100 mM NaNO₃. The electrodes were then tested again in an I-Cell with a pH 7.15 phosphate buffer solution containing 6 M NaNO₃ and 100 mM pyruvate to determine if the oligomycin had any inhibition effect on the mitochondria. Finally the electrodes were soaked for 12 hours in a pH 7.15 phosphate buffer solution that contained 100 mM NaNO₃ and 0.2 μL/mL nitrobenzene. The electrodes were tested again in an I-Cell with a pH 7.15 phosphate buffer solution containing 6 M NaNO₃ and 100 mM pyruvate to determine if the nitrobenzene had any activating effects on the inhibited mitochondria.

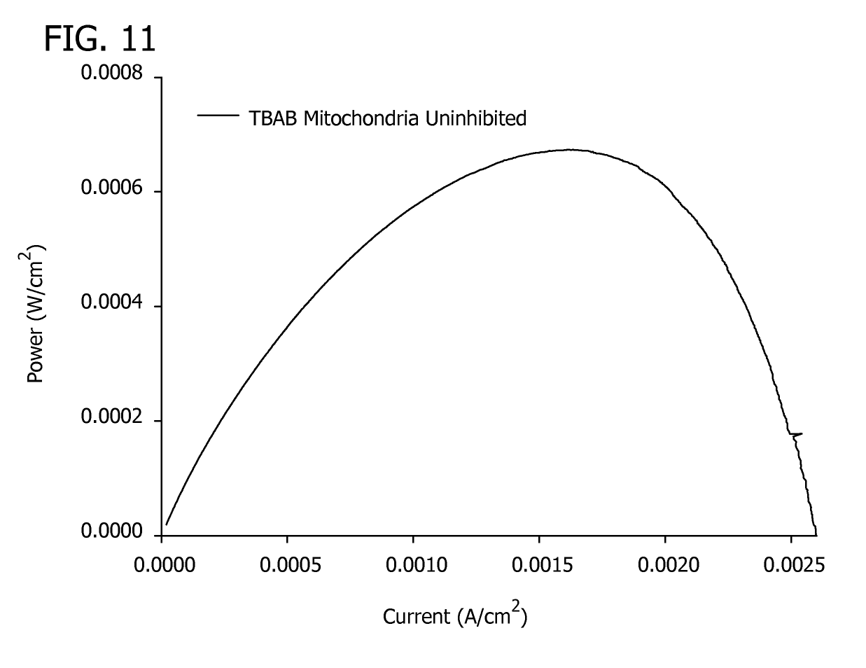

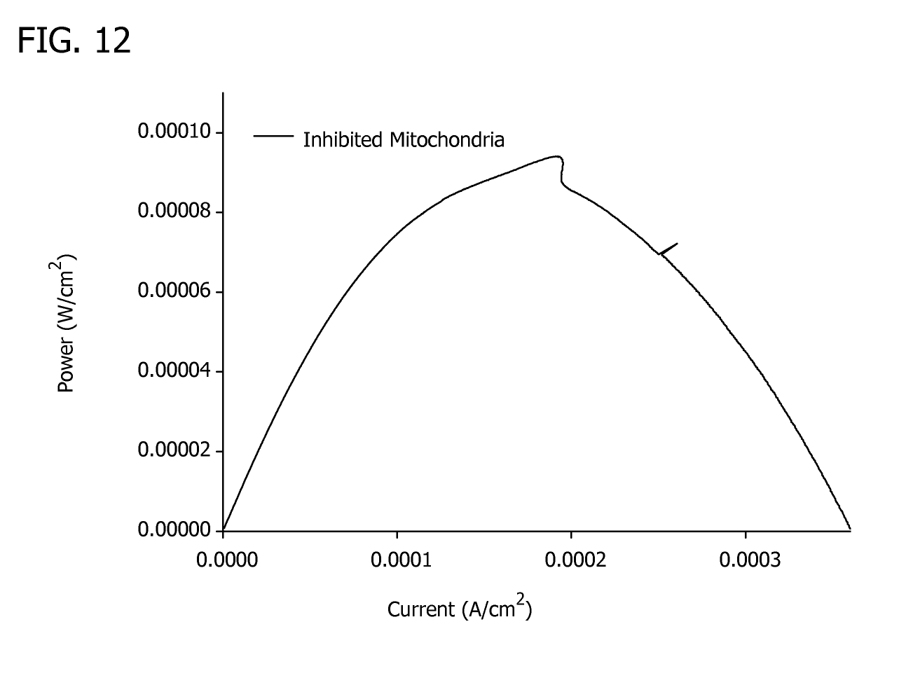

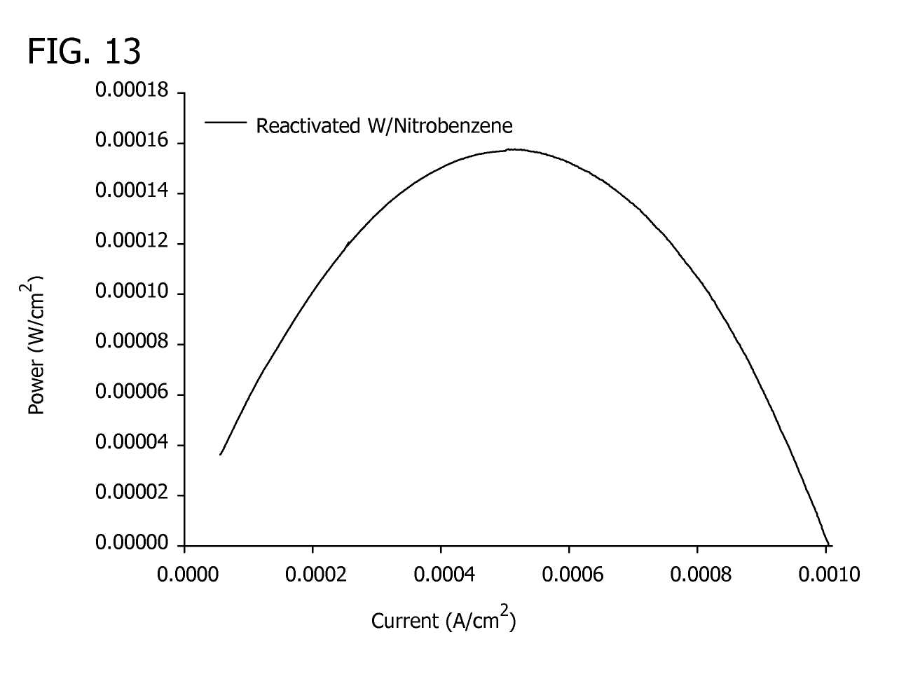

The initial electrodes' uninhibited performance showed average open circuit potential of 1.02±0.01 Volts and an average maximum current of 2.629±0.043 mA/cm². A representative power curve is shown in FIG. 11. Once soaked in the oligomycin solution, the performance declined (mitochondrial activity was inhibited by oligomycin, so it turned off the majority of the power of the biofuel cell). The average open circuit potential was 1.05±0.01 Volts. The average maximum current was 0.651±0.254 mA/cm². A representative power curve is shown in FIG. 12. The mitochondria performance did increase again after soaking in the nitrobenzene solution. The average open circuit potential was 1.06±0.05 Volts. The average maximum current was 0.897±0.134 mA/cm² and a representative power curve is shown in FIG. 13. These results show that a mitochondrial-based bioanode can be used in a self-powered sensor for detecting nitroaromatic explosives, because the power output after mitochondria inhibition is too low to power signaling electronics, but the power after exposure to a nitroaromatic is sufficient to power signaling electronics.

The oligomycin did show an average activity of 25% of the initial uninhibited performance of the mitochondria-modified electrode, thus, showing that oligomycin can inhibit the mitochondrial activity and decrease the power performance of the biofuel cell. The reactivation of the mitochondria with the nitrobenzene showed a 40% increase in activity compared to the oligomycin, which is sufficient for use as a self-powered sensor device.

### Example 7

### Immobilized Thylakoids

Thylakoid isolation. The thylakoids were isolated by homogenizing deveined spinach in 30 mM Tricine buffer (pH 7.7) with 0.3 M NaCl and 3 mM MgCl₂ with a chilled kitchen blender at 5° C. The resulting homogenate was filtered and 35 mL of the dark green filtrate was placed in a 40 mL centrifuge tube and centrifuged at 2500×g for 4 minutes at 4° C. The supernatant was discarded. The dark green pellet was washed in 20 mM Tricine buffer (pH 7.5) with 0.2 M sucrose and 3 mM MgCl₂ and 10 mM KCl. The suspension was centrifuged at 1000×G for 20 seconds to remove crude cell debris and other organelles. The supernatant was centrifuged at 5000×G for 10 minutes. The resulting pellet was washed again following the above procedure and then resuspended in 5 mL of the 20 mM Tricine buffer (pH 7.5) with 0.2 M sucrose and 3 mM MgCl₂ and 10 mM KCl.

The thylakoid suspension was then combined with either TBAB modified Nafion® or 2 wt. % of hexanal modified chitosan suspended in t-amyl alcohol as described in Example 6.

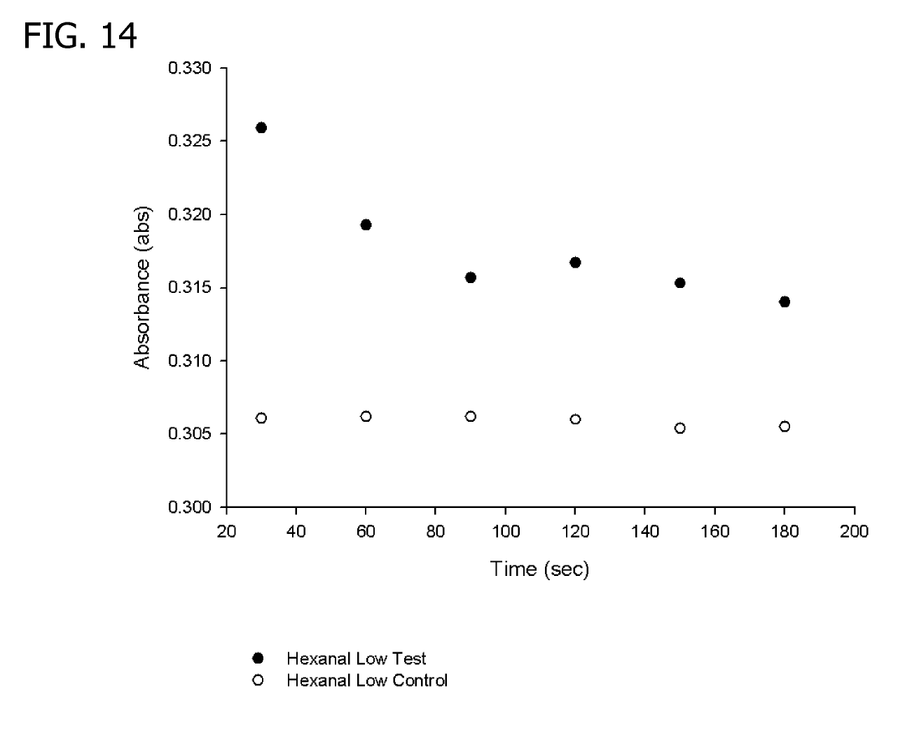

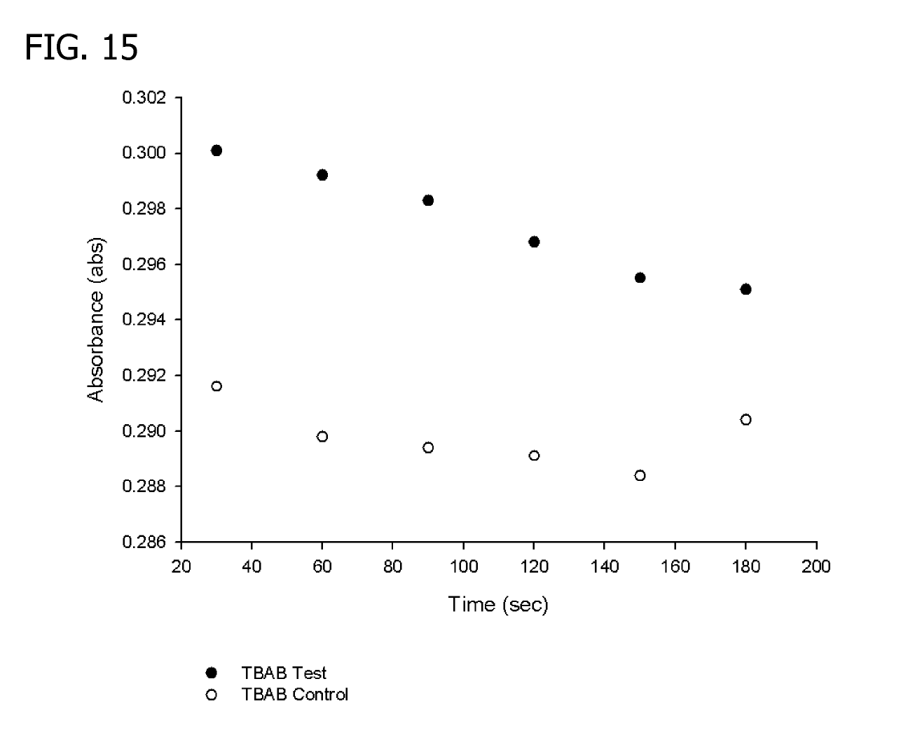

Assay procedure. Thylakoid activity was measured by the formation of NADPH. Dichlorophenolindophenol (DCPIP) was used the chromophor for measuring activity. 2.5 mL of the buffer and 200 microliters of 500 micromolar DCPIP solution were added to cuvettes that had been either coated with the polymer (control) or co-cast with the polymer and the thylakoid membrane suspension. Absorbance was measured at 620 nm after different times of illumination with bright light. Graphs of absorbance vs. time for the thylakoids immobilized in hexanal modified chitosan and TBAB-modified Nafion® are represented in FIGS. 14 and 15, respectively.

### Example 8

### Preparation of Alkyl Modified Chitosan

Medium molecular weight chitosan (available from Aldrich) (0.500 g) was dissolved by rapid stirring in 15 mL of 1% acetic acid. This resulted in a viscous gel-like solution and then 15 mL of methanol was added. The chitosan gel was allowed to stir for approximately 15 minutes, then 20 mL aldehyde (butanal, hexanal, octanal, or decanal) was added to the chitosan gel, followed by 1.25 g of sodium cyanoborohydride. The gel was continuously stirred until the suspension cooled to room temperature. The resulting product was separated by vacuum filtration and washed with 150 mL increments of methanol three times. The modified chitosan was then dried in a vacuum oven at 40° C. for two hours, leaving a flaky white solid. One percent by weight suspensions of each of the polymers were formed in 50% acetic acid, chloroform, and t-amyl alcohol.

### Example 9

### Preparation of Ru(bipyridine)₂(4-methyl-4′-(6-bromohexyl)-2,2′-bipyridine)⁺² Modified Chitosan

The preparation of Ru(bipyridine)₂(4-methyl-4′-(6-bromohexyl)-2,2′-bipyridine)⁺² modified chitosan started with the synthesis of a substituted bipyridine, 4-methyl-4′-(6-bromohexyl)-2,2′-bipyridine. To prepare the substituted bipyridine, 50 mL THF containing 1.69 g 4,4′-dimethyl-2,2′-bipyridine was added dropwise over 30 minutes to 4.1 mL of THF containing 9.1 mmol lithium diisopropylamine. This mixture was stirred for 1.5 hours, then cooled to 0° C., followed by dropwise addition of 9.2 mmol dibromoalkane of desired chain length with stirring. This mixture was stirred for 1.5 hours, quenched with ice water, and extracted with ether. The residue was recrystallized 3 times from ethyl acetate. Once the 4-methyl-4′-(6-bromohexyl)-2,2′-bipyridine was prepared, it was reacted to form the Ru(bipyridine)₂(4-methyl-4′-(6-bromohexyl)-2,2′-bipyridine)⁺² by refluxing 1.315 g of Ru(bpy)₂Cl₂(in its hydrate form), 0.8201 g of 4-methyl-4′-(6-bromohexyl)-2,2′-bipyridine, and 0.76 g sodium bicarbonate in 60 mL of 2:3 methanol-water solution until the Ru(bpy)₂Cl₂ was depleted. The depletion of Ru(bpy)₂Cl₂ was determined by UV-Vis absorption data. The resulting complex was precipitated by adding 4 mL of 3 M ammonium hexafluorophosphate (or a sodium or potassium perchlorate salt), followed by recrystallization from acetone/CH₂Cl₂. This reaction sequence yielded 77% Ru(bipyridine)₂(4-methyl-4′-(6-bromohexyl)-2,2′-bipyridine)⁺².

After its preparation, 137 mg Ru(bipyridine)₂(4-methyl-4′-(6-bromohexyl)-2,2′-bipyridine)⁺² was dissolved in a mixture of 5 mg of deacetylated chitosan in 1% acetic acid and DMF (1:1, 1 mL). This mixture was heated at 90° C. for 12 hours. After the reaction period, acetonitrile was added to precipitate Ru(bipyridine)₂(4-methyl-4′-(6-hexyl)-2,2′-bipyridine)⁺² modified chitosan. The precipitate was collected and purified by dissolution in 1% acetic acid, then recrystallized in methanol and dried under reduced pressure.

### Example 10

### Fluorescence Imaging of Hydrophobically Modified Chitosans

Two microliters of each polymer suspension were cast onto a glass microscope slide (Fisher) and dried in the desiccator. A 20 μL volume of 0.01 mM Ru(bpy)₃²⁺ or 0.01 mM FITC was pipetted onto the polymer cast and allowed to soak for two minutes. After soaking, the slides were rinsed with 18 MΩ water and allowed to dry in the desiccator. The polymers were imaged using an Olympus BX60M epifluorescence microscope (Melville, N.Y.). The polymers were observed under a 40× ultra-long working distance lens with a video camera (Sony SSC-DC50A). Fluorescence excitation was achieved with a mercury lamp. A frame grabber card (Integral Technologies, Inc., Indianapolis, Ind.) was used to acquire images, and the images were analyzed using SPOT software (Diagnostic Instruments, Inc.) on a Dell PC. Fluorescence imaging of each of the hydrophobically modified polyelectrolytes in Ru(bpy)₃⁺² and fluorescein was performed to determine the morphological effects of the hydrophobic modification. Representative fluorescence micrographs of hydrophobically modified chitosan in Ru(bpy)₃⁺² show that aggregates form within the hydrophobically modified chitosans and that the morphology changes with alkyl chain length. The butyl modified chitosan appeared to have small, fibrous interconnects, whereas the hexyl modified chitosan had large domains containing smaller micellar domains. As the alkyl chain length increased, the number of micellar domains decreased, but the size of the domain increased. Fluorescence micrographs of unmodified chitosan did not show distinct domains, so micellar structure was not observed for unmodified chitosan. Representative fluorescence micrographs of hydrophobically modified chitosan membranes soaked in FITC showed the same morphological changes were observed with either the cationic or the anionic fluorescent dye.

### Example 11

### Electrochemical Measurements of Hydrophobically Modified Chitosans

Glassy carbon working electrodes (3 mm in diameter, CH Instruments) were polished on a Buehler polishing cloth with 0.05 micron alumina and rinsed in 18 MΩ water. Two microliters of each polymer suspension was cast onto a glassy carbon electrode surface and allowed to dry in a vacuum desiccator until use. Cyclic voltammetry was used to measure the flux of the redox species through the polymer membrane at the electrode surface. The working electrodes were allowed to equilibrate in a 1.0 mM redox species solution containing 0.1 M sodium sulfate as the supporting electrolyte along with a platinum mesh counter electrode and measured against a saturated calomel reference electrode. The redox species studied were caffeine, potassium ferricyanide, and Ru(bpy)₃²⁺. The data were collected and analyzed on a Dell computer interfaced to a CH Instruments potentiostat model 810. Cyclic voltammetry was performed at scan rates ranging from 0.05 V/s to 0.20 V/s. All experiments were performed in triplicate and reported uncertainties correspond to one standard deviation.

Cyclic voltammetric studies of the two hydrophobically modified polyelectrolytes were conducted as a function of the alkyl chain length of the hydrophobic modification. All cyclic voltammetric experiments showed linear iₚ vs v^{1/2} plots, signifying transport-limited electrochemistry. Since electrochemical flux is a function of concentration as shown in Equation 2, KD^{1/2} values are reported in this paper as a concentration independent method of comparing fluxes.

$$Flux = \frac{i}{nFA} = \frac{2.69 \times {10}^{5} {n}^{3 / 2} AC * {v}^{1 / 2} {KD}^{1 / 2}}{nFA} \qquad Equation 2$$

where i is the peak current, n is the number of electrons transferred, F is Faraday's constant, A is the area of the electrode, C* is the concentration of redox species, v is the scan rate, K is the extraction coefficient, and D is the diffusion coefficient. The solvent determines the degree of swelling of the polymer during re-casting. Most literature studies on chitosan and chitosan derivatives employ acetic acid as the solvent for resuspension, however, it is important to note from the KD^{1/2} values for chloroform provides a higher average flux. Unmodified chitosan is only soluble in the acetic acid solution. The KD^{1/2} value for unmodified chitosan in caffeine is 5.52 (×0.14)×10⁻³. It is clear that hydrophobic modification of chitosan can decrease the flux of caffeine, but cannot make appreciable increases in flux.

On the other hand, transport of large, hydrophobic ions, like Ru(bpy)₃⁺², can be greatly affected by small changes in pore structure/size. The KD^{1/2} value for Ru(bpy)₃⁺² transport through unmodified chitosan is 2.17 (±0.33)×10⁻⁴. It is evident that hydrophobic modification of chitosan increases the transport of Ru(bpy)₃⁺² in all cases, by as much as 11.1 fold for octyl modified chitosan membrane resuspended in t-amyl alcohol.

### Example 12

### Preparation of Electrodes

A solution of 2 wt. % of a hydrophobically modified chitosan polymer was suspended in t-amyl alcohol and a solution of glucose oxidase was added. This solution was pipetted onto an electrode material. This electrode material was typically a carbon cloth, or other carbon material.

### Example 13

### Glucose Oxidase Activity Tests for Hydrophobically Modified Chitosans

Glucose oxidase (GOx) catalyzes the oxidation of β-D-glucose to D-glucono-δ-lactone with the concurrent release of hydrogen peroxide. It is highly specific for β-D-glucose and does not act on α-D-glucose. In the presence of peroxidase, hydrogen peroxide enters into a second reaction in the assay involving p-hydroxybenzoic acid and 4-amino antipyrine with the quantitative formation of quinoneimine dye complex, which is measured at 510 nm. The activity of GOx enzyme was measured in each of the hydrophobically modified Nafion® and chitosan membranes. The absorbance was measured at 510 nm against water after immobilizing the GOx enzyme within the hydrophobically modified chitosan membranes, and casting it in a plastic vial. All experiments were performed in triplicate and reported uncertainties correspond to one standard deviation.

The highest enzyme activity was observed for glucose oxidase in a hexyl modified chitosan suspended in t-amyl alcohol. These immobilization membranes showed a 2.53 fold increase in GOx enzyme activity over enzyme in buffer.

### Example 14

### Biofuel Cell

A biofuel cell having a bioanode comprising mitochondria immobilized in a hydrophobically modified chitosan is prepared by mixture casting a hydrophobically modified chitosan with a solution of mitochondria and buffer and pipetting the mixture on a carbon cloth, thus, forming a bioanode. A biocathode comprising an enzyme immobilized in a hydrophobically modified Nafion® membrane can be used to form a biofuel cell having a bioanode and a biocathode. Alternatively, a biofuel cell having a cathode comprising a chloroplast and/or a thylakoid immobilized in a hydrophobically modified chitosan or hydrophobically modified Nafion® membrane is prepared by mixture casting a hydrophobically modified chitosan or a hydrophobically modified Nafion® membrane with a solution of chloroplast and buffer and pipetting the mixture on a carbon paper or other electron conductor, thus, forming a biocathode.

### Example 15

### Microfluidic Biofuel Cell

Masters for the production of PDMS micromolding channels are made by coating a 4-in. silicon wafer with SU-8 10 negative photoresist using a spin coater (Brewer Science, Rolla, Mo.) operating with a spin program of 1000 rpm for 30 seconds for micromolding channel. For flow channels, a spin program of 1750 rpm for 30 seconds is used with SU-8 50 negative photoresist. The photoresist is prebaked at 90° C. for 5 minutes prior to UV exposure for 4 minutes with a near-UV flood source (Autoflood 1000, Optical Associates, Milpitas, Calif.) through a negative film containing the micromolding channel or flow channel design structures (Jostens, Topeka, Kans.). The transparency is made from a computer design drawn in Freehand (PC Version 8.0, Macromedia Inc., San Francisco, Calif.). The design is transferred to a transparency using an image setter with a resolution of 2400 dpi by a printing service (Jostens, Topeka, Kans.). Following this exposure, the wafer is postbaked at 90° C. for 5 minutes and developed in Nano SU-8 developer. The wafers containing the desired design are rinsed with acetone and isopropanol in order to remove any excess, unexposed photoresist that may have remained on the silicon wafer. The thickness of the photoresist is measured with a profilometer (Alpha Step-200, Tencor Instruments, Mountain View, Calif.), which corresponds to the channel depth of the PDMS structures.

A degassed 10:1 mixture of Sylgard 184 elastomer and curing agent are then poured onto the silicon wafer and cured at 75° C. for approximately 2 hrs. The PDMS is removed from the master wafer by cutting around the edges and peeling back the PDMS from the wafer. The master could be reused in order to generate numerous copies of the PDMS channels. The resulting PDMS flow channel is 200 mm wide, 100 mm deep and 3.0 cm long.

Soda-lime glass plates are purchased from a local glass shop. The plates are 7 cm wide, 10 cm long and 1.54 mm thick. The glass plates are cleaned by soaking them for 15 minutes in piranha solution (70% concentrated H₂SO₄/30% H₂O₂) to remove organic impurities. Glass is then rinsed thoroughly with Nanopure (18 MΩ-cm) water and dried with nitrogen. Using traditional lithographic and sputtering procedures, palladium electrodes are fabricated on the glass in specific patterns. Each plate could hold several flow channels with electrodes. This is more specifically accomplished by argon ion sputtering of a layer of titanium, for adhesive properties, and a layer of palladium. In order to accomplish this, the glass is placed into a deposition system (Thin Film Deposition System, Kurt J. Lesker Co.) for deposits of the metals. The thickness of the metals is monitored using a quartz crystal deposition monitor (Inficon XTM/2, Leybold Inficon). Titanium is deposited from a Ti-target at a rate of ˜2.3 angstroms/s to a depth of 200 angstroms. Palladium is deposited from a Pd-target at a rate of ˜1.9 angstroms/s to a depth of 2000 angstroms. AZ 1518 positive photoresist is dynamically dispensed onto the palladium coated glass. A pre-exposure bake at 95° C. for 1 minute is followed by a 9 second ultra-violet exposure through a positive film. The film is removed and the glass placed in a commercially available developer (AZ 300 MIF developer) for 45 seconds. After rinsing with water and drying with nitrogen, the glass is post baked for 1 minute at 95° C. Wet etching is employed using Aqua regia (8:7:1 H₂O:HCl:HNO₃) to remove the unwanted palladium and a titanium etchant to remove unwanted titanium from the glass. Once completed, the glass is rinsed with acetone and isopropanol to remove the remaining photoresist and dried with nitrogen.

A flow access hole is drilled through each glass plate, while immersed under water, with a 1-mm diamond drill bit and a Dremel rotary tool (Dremel). The syringe connector portion of a leur adapter is removed with the Dremel rotary tool and accompanying cutting disc. After polishing with a sanding disc, the leur adapter is affixed to the glass plate with J. B. Weld. The epoxy is cured in an oven (75° C.) for 2 hours before use. Connections are made to the palladium electrodes by copper wire and colloidal silver.

To fabricate carbon ink microelectrodes, first the PDMS micromolding channel is sealed to the glass plate in contact with the palladium leads (with leur fitting attached) that had been thoroughly cleaned. The PDMS channels are first primed with solvent thinner (N-160). The thinner is removed by applying a vacuum to one of the reservoirs. As soon as the thinner had been removed, a mixture of commercially available carbon ink and solvent thinner is added to the channels and pulled through the channel by applying vacuum (via water aspirator) to the opposite end. The ink/thinner mixture is made so that the volume of added thinner is 0.2% (v/w) of the initial ink weight. After filling channels with carbon ink, the reservoir where vacuum had been applied is filled with the ink/thinner solution and the entire chip placed in an oven at 75° C. for one hour. After this period of time, the PDMS could be removed from the glass, leaving the carbon microelectrode attached to the glass surface. A final curing/conditioning step is achieved by placing the chip in a separate oven at 12° C. for one hour. The height of the carbon microelectrode is measured with a profilometer and the width is measured via microscopy.

In order to further characterize the carbon ink electrodes, cyclic voltammetry is employed and performed in a 3-electrode format using a CH Instruments 810 bipotentiostat (Austin, Tex.). The carbon microelectrode is the working electrode with a silver/silver chloride reference electrode and a platinum wire as the auxiliary electrode. A static cell for cyclic voltammetry experiments is created in a piece of PDMS by cutting a small section (1 cm×2 cm) out of a larger piece of PDMS (2 cm×3 cm); this piece of PDMS is then sealed over the carbon electrode so the entire length of the electrode is exposed to solution. For flow experiments, a PDMS microchannel (˜200 mm wide, 100 mm deep and 2 cm long) is sealed over the carbon electrode, so the entire electrode is sealed inside the microchannel. The auxiliary and reference electrodes are contained in the outlet reservoir by use of an electrochemical cell holder (CH Instruments).

The flow access hole drilled in the glass plate allows for access to flow from a syringe pump (Pump 11, Harvard Apparatus, Holliston, Mass.). A syringe is filled with the solution of choice and placed in the syringe pump. With the use of high pressure fittings, leur adapters, and Teflon PEEK tubing, the syringe is connected to the glass microchip. The flow rates are varied from 0 μL/min to 15 μL/min through the 200 μm-wide PDMS flow channel which is aligned with one end at the flow access hole. The channel is sealed directly over the electrode. At the other end of the channel, a reservoir is formed by a hole punch and is where the cathode or reference and counter electrodes are placed.

The carbon ink electrode generally is a 2.5 cm long electrode that is 55 μm wide and 87 μm high. A solution of 1 mM tris(2,2′-bipyridyl)dichlororuthenium(II) hexahydrate and 0.1M sodium sulfate as the electrolyte is used to characterize the response of the electrode using cyclic voltammetry. As flow rate is increased, the current density increased which is expected due to the analyte reaching the electrode surface faster with an increase in flow rates. Initially, an electrochemical pretreatment is utilized to clean the electrode by applying 1.5 V for 3 minutes in a 0.05 M phosphate buffer (pH 7.4).

The procedure above is followed with slight modification to simplify the process of forming an electrode comprising an electron conductor and an enzyme or organelle immobilization material. To do so, the electron conductor solution is modified to include the enzyme or organelle immobilization material. The additional material is prepared by adding a 2 wt. % solution of a hydrophobically modified chitosan in t-amyl alcohol is suspended in Ercon N160 Solvent Thinner and vortexed thoroughly. Alternatively, the additional material is prepared by adding 0.0003 moles of TBAB to 1 mL of Nafion® in a weigh boat and allowing the mixture to air dry. After drying, water is added to rinse the mixture, and the mixture is allowed to air dry overnight. Next, the mixture is rinsed two more times with water and allowed to air dry. Then the material is suspended in 1 mL of Ercon N 160 Solvent Thinner and vortexed thoroughly. Finally, 1 mL of either of these modified thinners is added to 0.5 g Ercon E-978(I) carbon-based ink. This modified electron conductor solution is then flowed through the mold cavity formed by the casting mold and the substrate and cured according to the method described above in this example.

To form a bioanode according to the invention, the general steps above in this example are used, with the anode being completed by flowing additional materials over the electron conductor after its curing and activation stages. A casting solution of the remaining anode elements is created by combining a 2 wt. % solution of hydrophobically modified chitosan in t-amyl alcohol, and a mitochondria solution in lower aliphatic alcohol. Alternatively, 100 mL of modified Nafion® and about 200 mL mitochondria solution is combined in a lower aliphatic alcohol. Either of these solutions is then vortexed together thoroughly and pumped through the approximately 100 mm microchannel at a flow rate of about 1 mL/min. The electron conductor and the casting solution are then allowed to dry overnight.

For the biocathode, the microchips and channel masters are fabricated as described above in this example using photolithography. The carbon ink microelectrodes generated from the micromolding procedure could be further modified with the hydrophobically modified chitosan membrane mixture described above.

The carbon microelectrodes are modified to serve as a bioanode. A hole is punched in PDMS to form a bulk reservoir that is placed around the microelectrode and include Ag/AgCl reference electrode and a platinum wire as the auxiliary electrode. Specifically, this is a static cell.

The mitochondria/hydrophobically modified chitosan mixture or mitochondria/modified Nafion® is immobilized onto the carbon microelectrode using microchannels that are reversibly sealed over the microelectrodes and hydrodynamic flow. The size of this flow channel is such that alignment over the microelectrode is possible but is not much wider than the electrode. To accomplish this, a PDMS microchannel (130 mm wide, 100 mm deep and ˜2 cm long) is sealed over the carbon electrode (˜40 mm wide, ˜2 cm long, and ˜100 mm high), so that the entire electrode is sealed inside the microchannel. A 2:1 ratio of mitochondria and modified Nafion® or hydrophobically modified chitosan mixture is prepared and vortexed until sufficiently mixed. The mixture is introduced to the channels thru a syringe by use of a syringe pump (Harvard Apparatus, Brookfield, Ohio) at 1.0 mL/min. Once the mixture travels the entire length of the channel (monitored visually), the solvent is allowed to evaporate at room temperature. This is possible since PDMS is permeable to gases. After evaporation is complete, the PDMS is removed, leaving a coated bioanode.

To form a biocathode according to the invention, the general steps described in this example are used, with the biocathode being completed by flowing additional materials over the electron conductor after its curing and activation stages.

To modify the electron conductor, a casting solution of cathode enzyme or cathode organelle, and a hydrophobically modified chitosan is vortexed together for about 20 minutes. Alternatively, a casting solution of cathode enzyme or cathode organelle, and a hydrophobically modified Nafion® membrane is vortexed together for about 20 minutes. Next, the solution is pumped through the approximately 100 mm microchannel at a flow rate of about 1 mL/min. The electron conductor and the casting solution are then allowed to dry overnight.

The biocathode is created in a similar fashion to the bioanode described above. A PDMS microchannel is sealed over a carbon ink microelectrode. Hydrophobically modified chitosan is mixed with a cathode organelle. Specifically, the hydrophobically modified chitosan is mixed with a chloroplast. The mixture is then pumped through the channel at a 1.0 mL/min until it reached the end of the channel after which time the solvent is allowed to evaporate. Afterwards the PDMS flow channel is removed leaving a coated electrode that is used as a biocathode.

In view of the above, it will be seen that the several objects of the invention are achieved and other advantageous results attained.

As various changes could be made in the above methods without departing from the scope of the invention, it is intended that all matter contained in the above description or shown in the accompanying drawings shall be interpreted as illustrative and not in a limiting sense.

Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

1. A bioelectrode comprising
an electron conductor;
at least one organelle comprising at least one enzyme; and
an organelle immobilization material capable of immobilizing the organelle;
wherein either the organelle is isolated from a cell or the organelle immobilization material is non-microbial.

2. The bioelectrode of claim 1 wherein the bioelectrode is a bioanode and either:
(a) the enzyme is capable of reacting with a fuel fluid to produce an oxidized form of the fuel fluid, the enzyme being capable of releasing electrons to the electron conductor, and the organelle immobilization material being permeable to the fuel fluid;
(b) the enzyme is capable of reacting with an oxidized form of an electron mediator and a fuel fluid to produce an oxidized form of the fuel fluid and a reduced form of the electron mediator, the reduced form of the electron mediator being capable of releasing electrons to the electron conductor, and the organelle immobilization material being permeable to the fuel fluid; or
(c) the enzyme is capable of reacting with an oxidized form of an electron mediator and a fuel fluid to produce an oxidized form of the fuel fluid and a reduced form of the electron mediator, the organelle immobilization material being permeable to the fuel fluid, and the bioanode further comprises an electrocatalyst adjacent the electron conductor, an oxidized form of the electrocatalyst being capable of reacting with the reduced form of the electron mediator to produce an oxidized form of the electron mediator and a reduced form of the electrocatalyst, the reduced form of the electrocatalyst being capable of releasing electrons to the electron conductor.

3. The bioelectrode of claim 1 wherein the bioelectrode is a biocathode and either:
(a) the enzyme is capable of reacting with an oxidant to produce water, the enzyme being capable of gaining electrons from the electron conductor, the organelle immobilization material being permeable to the oxidant;
(b) the enzyme is capable of reacting with a reduced form of an electron mediator and an oxidant to produce an oxidized form of the electron mediator and water, the oxidized form of the electron mediator being capable of gaining electrons from the electron conductor to produce the reduced form of the electron mediator, and the organelle immobilization material being permeable to the oxidant; or
(c) the enzyme is capable of reacting with a reduced form of an electron mediator and an oxidant to produce an oxidized form of the electron mediator and water, the organelle immobilization material being permeable to the oxidant, and the biocathode further comprising an electrocatalyst adjacent the electron conductor, an oxidized form of the electrocatalyst being capable of gaining electrons from the electron conductor to produce a reduced form of the electrocatalyst that is capable of reacting with an oxidized form of the electron mediator to produce a reduced form of the electron mediator and an oxidized form of the electrocatalyst.

4-52. (canceled)

53. The bioelectrode of claim 1 wherein the organelle immobilization material comprises a micellar or inverted micellar structure.

54. The bioelectrode of claim 53 wherein the organelle immobilization material comprises perfluoro sulfonic acid-polytetrafluoro ethylene (PTFE) copolymer; modified perfluoro sulfonic acid-polytetrafluoro ethylene (PTFE) copolymer; polysulfone; a micellar polymer; a poly(ethylene oxide) based block copolymer; a polymer formed from microemulsion and/or micellar polymerization; copolymers of alkyl methacrylates, alkyl acrylates, and styrenes; ceramics; sodium bis(2-ethylhexyl)sulfosuccinate; sodium dioctylsulfosuccinate; lipids; phospholipids; sodium dodecyl sulfate; decyltrimethylammonium bromide; tetradecyltrimethylammonium bromide; (4-[(2-hydroxyl-1-naphthalenyl)azo]benzenesulfonic acid monosodium salt); linoleic acids; linolenic acids; colloids; liposomes; micelle networks; and combinations thereof.

55. The bioelectrode of claim 54 wherein the organelle immobilization material comprises a modified perfluoro sulfonic acid-PTFE copolymer wherein the modified perfluoro sulfonic acid-PTFE copolymer is modified with a hydrophobic cation larger than NH₄⁺.

56. The bioelectrode of claim 55 wherein the hydrophobic cation comprises a quaternary ammonium ion represented by formula 1 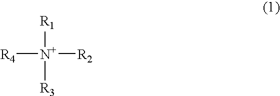
wherein R₁, R₂, R₃ and R₄ are independently hydrogen, hydrocarbyl, substituted hydrocarbyl or heterocyclo wherein at least one of R₁, R₂, R₃ and R₄ is other than hydrogen.

57. The bioelectrode of claim 56 wherein either: R₁, R₂, R₃ and R₄ are independently hydrogen, methyl, ethyl, propyl, butyl, pentyl, hexyl, heptyl, octyl, nonyl or decyl wherein at least one of R₁, R₂, R₃ and R₄ is other than hydrogen; R₁, R₂, R₃ and R₄ are the same and are methyl, ethyl, propyl, butyl, pentyl or hexyl; R₁, R₂, R₃ and R₄ are butyl; or one of R₁, R₂, R₃, and R₄ is hexyl, octyl, decyl, dodecyl, or tetradecyl and the others are independently methyl, ethyl, or propyl.

58. The bioelectrode of claim 1 wherein the organelle immobilization material is a hydrophobically-modified polysaccharide comprising chitosan, cellulose, chitin, starch, amylose, or a combination thereof.

59. The bioelectrode of claim 58 wherein the polysaccharide corresponds to Formula 2 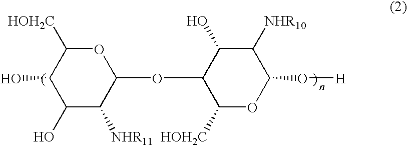
wherein n is an integer;
R₁₀ is independently hydrogen, hydrocarbyl, substituted hydrocarbyl, or a hydrophobic redox mediator; and
R₁₁ is independently hydrogen, hydrocarbyl, substituted hydrocarbyl, or a hydrophobic redox mediator.

60. The bioelectrode of claim 59 wherein either: R₁₀ is independently hydrogen or alkyl and R₁₁ is independently hydrogen or alkyl; R₁₀ is independently hydrogen or hexyl and R₁₀ is independently hydrogen or hexyl; R₁₀ is independently hydrogen or octyl and R₁₁ is independently hydrogen or octyl; R₁₀ is independently hydrogen or a hydrophobic redox mediator and R₁₁ is independently hydrogen or a hydrophobic redox mediator.

61. The bioelectrode of claim 60 wherein the hydrophobic redox mediator is a transition metal complex of osmium, ruthenium, iron, nickel, rhodium, rhenium, or cobalt with 1,10-phenanthroline (phen), 2,2′-bipyridine (bpy) or 2,2′,2″-terpyridine (terpy), methylene green, methylene blue, poly(methylene green), poly(methylene blue), luminol, a nitro-fluorenone derivative, an azine, osmium phenanthrolinedione, catechol-pendant terpyridine, toluene blue, cresyl blue, nile blue, neutral red, a phenazine derivative, tionin, azure A, azure B, azure C, toluidine blue O, acetophenone, a metallophthalocyanine, nile blue A, modified transition metal ligand, 1,10-phenanthroline-5,6-dione, 1,10-phenanthroline-5,6-diol, [Re(phen-dione)(CO)₃Cl], [Re(phen-dione)₃](PF₆)₂, poly(metallophthalocyanine), poly(thionine), a quinone, a diimine, a diaminobenzene, a diaminopyridine, phenothiazine, phenoxazine, toluidine blue, brilliant cresyl blue, 3,4-dihydroxybenzaldehyde, poly(acrylic acid), poly(azure I), poly(nile blue A), polyaniline, polypyridine, polypyrole, polythiophene, poly(thieno[3,4-b]thiophene), poly(3-hexylthiophene), poly(3,4-ethylenedioxypyrrole), poly(isothianaphthene), poly(3,4-ethylenedioxythiophene), poly(difluoroacetylene), poly(4-dicyanomethylene-4H-cyclopenta[2,1-b;3,4-b′]dithiophene), poly(3-(4-fluorophenyl)thiophene), poly(neutral red), or a combination thereof.

62. The bioelectrode of claim 1 wherein the electron conductor comprises a carbon-based material, a metallic conductor, a semiconductor, a metal oxide, a modified conductor, or a combination thereof.

63. The bioelectrode of claim 62 wherein the electron conductor comprises carbon cloth, carbon paper, carbon screen printed electrodes, carbon black, carbon powder, carbon fiber, single-walled carbon nanotubes, double-walled carbon nanotubes, multi-walled carbon nanotubes, carbon nanotube arrays, diamond-coated conductors, glass carbon, mesoporous carbon, graphite, uncompressed graphite worms, delaminated purified flake graphite, high performance graphite, highly ordered pyrolytic graphite, pyrolytic graphite, polycrystalline graphite, or a combination thereof.

64. The bioelectrode of claim 3 wherein the organelle comprises hydrogenosome, chloroplast, or thylakoids.

65. The bioelectrode of claim 2 wherein the organelle comprises mitochondria, mitoplasts, peroxisome, or glyoxysome.

66. An organelle immobilized in an immobilization material capable of immobilizing the organelle, the material being permeable to a compound smaller than the organelle, wherein the immobilization material is either a non-naturally occurring colloidal material, an acellular colloidal material, a micellar material, or an inverted micellar material.

67. A biofuel cell for generating electricity comprising a fuel fluid, a bioanode of claim 2 and a cathode.

68. The biofuel cell of claim 67 wherein the organelle is mitochondria, the fuel fluid is pyruvate, and the bioanode further comprises an agent which inhibits the enzyme from reacting with the fuel fluid until the mitochondria is exposed to a nitroaromatic explosive; an alarm signal being produced when the nitroaromatic explosive is present; and an alarm that detects the alarm signal and provides an alert to the presence of the explosive.

69. A method for detecting a nitroaromatic explosive using the biofuel cell of claim 68 comprising exposing the biofuel cell to the nitroaromatic explosive so that the enzyme will react with the fuel fluid and the biofuel cell will generate electricity to produce the alarm signal indicating that the nitroaromatic explosive has been detected.

70. A bioanode comprising
(a) an electron conductor;
(b) at least one enzyme capable of reacting with a fuel fluid to produce an oxidized form of the fuel fluid, the enzyme either being capable of releasing electrons to the electron conductor or capable of releasing electrons to an electron mediator; and
(c) an enzyme immobilization material capable of immobilizing and stabilizing the enzyme, the material being permeable to the fuel fluid;
wherein the enzyme comprises a glycolysis enzyme, a Kreb's cycle enzyme, or a combination thereof.
